(19) United States
(12) Patent Application Publication
(10) Pub. No.: US 2026/0318919 A1
(43) Pub. Date: Oct. 15, 2026

(54) USING MASS SPECTROMETRY TO IDENTIFY ENDOMETRIOSIS TISSUE

(71) Applicant: Board of Regents, The University of Texas System, Austin, TX (US)

(72) Inventors: Livia Schiavinato Eberlin, Austin, TX (US); Michael T. Breen, Austin, TX (US); Jialing Zhang, Austin, TX (US); Clara L. Feider, Austin, TX (US); John Lin, Austin, TX (US); Thomas Milner, Austin, TX (US)

(21) Appl. No.: 19/463,313

(22) Filed: Jan. 29, 2026

Related U.S. Application Data
(63) Continuation of application No. 17/457,800, filed on Dec. 6, 2021, now abandoned.
Application No. 17/457,800 is a continuation of application No. PCT/US2020/036351, filed on Jun. 5, 2020.
(60) Provisional application No. 62/858,300, filed on Jun. 6, 2019.

(51) Int. Cl. G01N 33/68; G16H 50/20

(52) U.S. Cl. CPC G01N 33/6848; G16H 50/20; G01N 2560/00; G01N 2800/364

(57) ABSTRACT

In a general aspect, methods and devices are provided for using mass spectrometry to identify endometriosis. In some aspects, a fixed or discrete volume of a solvent is applied to a tissue site including possible endometriosis tissue. The applied solvent is collected to obtain a liquid sample. The liquid sample is subjected to mass spectrometry analysis. The liquid sample is collected from a tissue site in vivo during a medical procedure. The mass spectrometry data are analyzed to identify whether the tissue site comprises endometriosis.

### CROSS-REFERENCE TO RELATED APPLICATIONS

This application is a continuation of U.S. application Ser. No. 17/457,800 filed Dec. 6, 2021, which is a continuation of International Application No. PCT/US2020/036351 filed Jun. 5, 2020, which claims priority to U.S. Provisional Application No. 62/858,300 filed Jun. 6, 2019 which is hereby incorporated herein by reference in its entirety.

### BACKGROUND

The following description relates to using mass spectrometry to identify endometriosis tissue.

Tissue evaluation is critical in the diagnosis and management of patients. Currently, pathological evaluation of tissues removed during endometriosis surgeries occurs most of the times post-operatively using formalin fixed paraffin embedded tissues, a process that can provide definitive diagnosis typically in about 2 weeks. Because of the lack of accurate and fast intra-operative evaluation of tissue samples during endometriosis surgeries, endometriosis tissue can sometimes be left in the body and cause recurrence of the disease, which requires a follow-up surgery to further remove the endometriosis tissue.

### BRIEF DESCRIPTION OF THE DRAWINGS

FIG. 1 is a schematic diagram of an example system.

FIG. 2 is a schematic diagram showing aspects of an example system.

FIG. 3 is a schematic diagram showing aspects of an example sampling probe 300.

FIG. 4 is a flow diagram showing an example process 400 for tissue analysis.

FIG. 5A is an example optical image of an in-vivo endometriotic lesion.

FIG. 5B is an example mass spectrum of an ex-vivo endometriotic tissue sample taken from the cul-de-sac of a patient.

FIG. 6 are example mass spectra and respective post-analysis histopathological images of various tissue samples.

FIGS. 7A-7B are diagrams showing performance of a statistical classification model.

FIG. 8A is an example optical image of an in-vivo endometriosis lesion on a right ovary of a patient.

FIG. 8B are example mass spectra collected on an ex-vivo endometriosis tissue sample taken from a right ovary.

FIG. 9 is a block diagram showing aspects of an example system 900.

FIG. 10A are example mass spectra collected on an ex-vivo endometriosis tissue sample.

FIG. 10B is a diagram showing performance of a statistical classification model.

FIG. 11 is a diagram showing performance of a statistical classification model.

### DETAILED DESCRIPTION

The present description relates concerns methods and devices for assessment of tissue samples using mass spectrometry. Molecular approaches can provide highly accurate and potentially real-time assessments of tissue samples. Coupling the molecular approaches with minimally invasive surgical techniques, or non-invasive techniques can provide a highly accurate, yet low trauma, way to assess and diagnose tissue and surgical samples.

In a first embodiment there is provide a method for assessing tissue samples from a subject comprising: (a) applying a fixed or discrete volume of a solvent to a tissue site including possible endometriosis tissue in the subject; (b) collecting the applied solvent to obtain a liquid sample; and (c) subjecting the sample to mass spectrometry analysis. In some aspects, the method further comprises identifying the tissue site as endometriosis tissue versus healthy tissue. In certain aspects, the sample is collected in a substantially CO₂ atmosphere.

Still a further embodiment provides an apparatus for obtaining or producing samples (e.g., from tissues) for mass spectrometry analysis, the apparatus comprising: a chamber comprising a solvent; a gas supply (e.g., a pressurized gas supply); a mass spectrometer; a probe comprising a reservoir, a first conduit, a second conduit and a third conduit, wherein: the reservoir is in fluid communication with the first conduit, the second conduit and the third conduit; the first (solvent) conduit is in fluid communication with the chamber; the second (gas) conduit is in fluid communication with a gas supply; and the third (collection) conduit is in fluid communication with the mass spectrometer. In some aspects, the gas supply can be a pressurized gas supply. In some aspects, the probe is, or is comprised in, the cannula of a surgical instrument. In further aspects, the surgical instrument may be a laparoscope, trocar needle, biopsy guide, or multiple-lumen catheter. In certain aspects, the surgical instrument manually operated. In other aspects, the surgical instrument is robotic.

In yet still further aspects, the probe comprises a distal probe end and the distal probe end comprises a shutter that can be closed to prevent fluid communication outside of the probe. In some aspects, the shutter is a balloon that can be inflated to prevent fluid communication outside of the probe. In certain aspects, the balloon can be inflated with a gas or a liquid. In specific aspects, the shutter is a door that can be closed to prevent fluid communication outside of the probe. In other aspects, the shutter is configured such that is can be opened and closed multiple times. The shutter may be controlled manually or robotically. In several aspects, the first, second or third conduit is more than 1 meter in length. In additional aspects, the first conduit is in fluid communication with the third conduit; and the second conduit is in fluid communication with the third conduit. In further specific aspects, the first conduit is disposed within the third conduit. In other aspects, the second conduit is disposed within the third conduit.

In certain specific aspects, the first conduit and the second conduit are disposed within the third conduit. In further aspects, the first conduit comprises a first distal end; the second conduit comprises a second distal end; the third conduit comprises a third distal end; and the first distal end and the second distal end are located within the third conduit. In some aspects, the third distal end is located within the probe. In another aspect, the first distal end is located a first distance from the distal probe end; the second distal end is located a second distance from the distal probe end; the third distal end is located a third distance from the distal probe end; the first distance is greater than the third distance; and the second distance is greater than the third distance. In an additional aspect, the first distal end and the second distal end terminate proximal to a sample collection region of the third conduit. In certain aspects, the sample collection region is located between the first and second distal ends and the third distal end. In further specific aspects, the sample collection region is in fluid communication with the mass spectrometer via the third conduit. In some additional aspects, the apparatus further comprises a control system configured to control; a solvent flow from the chamber through the first conduit to the first distal end; a gas flow from the gas supply through the second conduit to the second distal end; and a sample flow through the third conduit to the mass spectrometer.

In yet still further aspects, the apparatus may additionally comprise a fourth conduit, wherein the first conduit, the second conduit and the third conduit are each in fluid communication with the fourth conduit. In some aspects, the apparatus may further comprise a first valve configured to control flow between the first conduit and the fourth conduit; and a second valve configured to control flow between the second conduit and the fourth conduit. In an additional aspect, the apparatus may further comprise a third first valve configured to control flow between the third conduit and the fourth conduit. In still additional aspects, the gas supply provides air, nitrogen or carbon dioxide to the probe. In certain aspects, the gas supply is a pressurized gas supply that provides a gas to the probe at a pressure between 0.1 psig and 5.0 psig. In other aspects, the pressurized gas supply provides a gas to the probe at a pressure between 0.5 psig and 2.5 psig. In specific aspects, the pressurized gas supply provides a gas to the probe at a pressure less than 100 psig. In some aspects, the gas for use in an apparatus of the embodiments may be provided by a pressurized gas supply. In further aspects, the gas can be pumped into an apparatus. Likewise, in some aspects, the gas can be pulled through an apparatus by use of a vacuum. In some aspects, the vacuum is provided by the mass spectrometer inlet. In further aspects, an additional vacuum system is employed. In certain aspects wherein the apparatus is used for a laparoscopic procedure, the gas supply can be a pressurized gas supply.

In some aspects, the solvent comprises water. In more specific aspects, the solvent comprises sterile water. In several aspects, the solvent comprises ethanol. In certain specific aspects, the solvent comprises an aqueous mixture including from 1 to 25% ethanol.

In still further aspects, the probe comprises a tracking device or dye to track a location of the probe. In additional aspects, the apparatus may further comprise a control system configured to control: a solvent flow from the chamber through the first conduit; a gas flow from the gas supply through the second conduit; and a sample flow through the third conduit to the mass spectrometer. In some aspects, the control system is configured to: control the solvent flow at a flow rate between 200 and 5000 microliters per minute for a period of time between 1 and 3 seconds; control the gas flow at a flow rate between 0.1 and 15 psig for a period of time between 5 and 50 seconds; and/or control the sample flow for a period of time between 5 and 50 seconds. In certain aspects, the control system comprises programing that initiates solvent flow.

In additional aspects, the mass spectrometer is in electronic communication with a computer that can provide sample analysis. In some aspects, the computer provides a visual or auditory read-out of the sample analysis. In further aspects, the apparatus may additionally comprise a waste container in fluid communication with the third conduit. In certain aspects, the apparatus may further comprise a valve configured to diverge a fluid from the third conduit to the waste container. In other aspects, the apparatus may further comprise a pump configured to remove contents of the waste container. In still further aspects, the apparatus may comprise a pump in fluid communication with the third conduit. In some aspects, the pump is configured to increase the velocity of the contents within the third conduit. In several aspects, the apparatus may further comprise a heating element coupled to the third conduit. In a specific aspect, the heating element is a heating wire.

In yet still further aspects, the apparatus may comprise an ionization device in fluid communication with the third conduit. In certain aspects, the ionization device is an electrospray ionization (ESI) device. In other aspects, the ionization device is an atmospheric pressure chemical ionization (APCI) device. In some aspects, the ionization device is to form a spray proximal to an inlet for mass spectrometer. In several aspects, the third conduit is not directly coupled to the mass spectrometer. In specific aspects, the apparatus may further comprise a venturi device in fluid communication with the third conduit. In certain aspects, the apparatus does not include device for application of ultrasonic or vibrational energy.

In a further embodiment there is provided a method for assessing tissue samples from a subject comprising (a) applying a fixed or discrete volume of a solvent to a tissue site in the subject through the cannula of a surgical instrument; (b) collecting the applied solvent to obtain a liquid sample; and (c) subjecting the sample to mass spectrometry analysis. In some aspects, the fixed or discrete volume of a solvent is not applied as a spray. In other aspects, the fixed or discrete volume of a solvent is applied as a droplet. In certain aspects, the surgical instrument is a laparoscope, trocar needle, or biopsy guide. The surgical instrument may be manually operated or robotic.

In further aspects, the cannulas comprised in a probe having a distal probe end and the distal probe end comprises a shutter that can be closed to prevent fluid from passing out of the cannula of the probe. In some aspects, the shutter is a balloon that can be inflated to prevent fluid communication outside of the probe. In specific aspects, the balloon can be inflated with a gas. In certain aspects, the shutter is a door than can be closed to prevent fluid communication outside of the probe. For example, the shutter can be an iris diaphragm, a mechanical closure, gate, or tapenade. In some aspects, the shutter can be manually controlled or may be automated. For example, in some aspects, the shutter may be on a timer that activates the shutter after solvent has been in contact with the tissue site for a predetermined time period (e.g., at least about 1, 2, or 3 seconds). In still further aspects, the fixed or discrete volume of a solvent is applied at using a pressure of less than 100 psig. In other aspects, the fixed or discrete volume of a solvent is applied at using a pressure of less than 10 psig. In some aspects, the fixed or discrete volume of a solvent is applied using a mechanical pump to move the solvent through a solvent conduit. In certain aspects, collecting the applied solvent comprises applying a negative pressure to pull the sample into a collection conduit and/or applying a gas pressure to push the sample into a collection conduit. In other aspects, collecting the applied solvent comprises applying a negative pressure to pull the sample into a collection conduit and applying a positive pressure to push the sample into a collection conduit. In certain specific aspects, the solvent is applied through a solvent conduit that is separate from the collection conduit. In further aspects, the gas pressure is applied through a gas conduit that is separate from the solvent conduit and the collection conduit. In still other aspects, applying a gas pressure to push the sample into a collection conduit comprises applying a pressure of less than 100 psig.

In yet still further aspects, the method produces no detectable physical damage to the tissue. In some aspects, the method does not involve application of ultrasonic or vibrational energy to the tissue. In certain aspects, the solvent may be sterile. In specific aspects, the solvent may be a pharmaceutically acceptable formulation, and further an aqueous solution, and still further sterile water. In further specific aspects, the solvent consists essentially of water. In other aspects, the solvent comprises from about 1 to 20% of an alcohol. In some aspects, the alcohol comprises ethanol. In still additional aspects, the discrete volume of solvent is between about 0.1 and 100 μL. In certain aspects, the discrete volume of solvent is between about 1 and 50 μL. In further aspects, collecting the applied solvent is between 0.1 and 30 seconds after the applying step. In another aspect, collecting the applied solvent is between 1 and 10 seconds after the applying step. In some aspects, the tissue site in an internal tissue site that is being surgically assessed.

In still further aspects, the method additionally comprises collecting a plurality liquid samples from a plurality of tissue sites. In certain aspects, the liquid samples are collected with a probe. In specific aspects, the probe is washed between collection of the different samples. In some aspects, the probe is disposable and is changed between collection of the different samples. In another aspect, the probe comprises a collection tip and further comprising ejecting the collection tip from the probe after the liquid samples are collected. In further aspects, the plurality of tissue sites comprises 2, 3, 4, 5, 6, 7, 8, 9 or 10 tissues sites. In an additional aspect, the plurality of tissue sites surrounds a section of tissue that has been surgically resected. In some aspects, the resected tissue is a tumor. In other aspects, the method is further defined as an intraoperative or post operative method. In certain aspects, the mass spectrometry comprises ambient ionization MS. In certain specific aspects, subjecting the sample to mass spectrometry analysis comprises determining a profile corresponding to the tissue site. In a further aspect, the method comprises comparing the profile to a reference profile to identify tissue sites that include diseased tissue. Still a further aspect comprises resecting tissue sites that are identified to include diseased tissue. In another aspect, the method is performed using an apparatus in accordance with the embodiments and aspects described above.

In further aspects, the mass spectrometer is in communication with a computer that provides a sample analysis. In certain aspects, the results of each sample analysis are provided by a visual or auditory output from the computer. For example, the results of each sample analysis by the computer can be indicated by a differently colored light that is illuminated or by a different frequency of sound produced. In some aspects, the mass spectrometer is a mobile the mass spectrometer. In further aspects, the mass spectrometer can comprise an uninterruptable power supply (e.g., a battery power supply). In still further aspects, the mass spectrometer comprises an inlet that may be closed to keep instrument vacuum. In yet further aspects, the mass spectrometer is separated from the probe by a mesh filter (e.g., to block contamination).

In some aspects, the reservoir is configured to form a droplet of the solvent. In certain aspects, the pressurized gas supply provides a gas to the probe at a pressure between 0.1 psig and 5.0 psig. In further aspects, the pressurized gas supply provides a gas to the probe at a pressure between 0.5 psig and 2.5 psig. In several aspects, the pressurized gas supply provides air to the probe. In other aspects, the pressurized gas supply provides an inert gas such as nitrogen or carbon dioxide to the probe. In some aspects, a gas supply for use according to the embodiments is at atmospheric pressure. For example, the conduit for delivery of gas may be supplied by the atmosphere around the apparatus.

In additional aspects, the apparatus further comprises a pump configured to transfer the solvent from the chamber to the first conduit. In further aspects, the apparatus may comprise a first valve configured to control a flow from the third conduit to the mass spectrometer. In some aspects, the third conduit is under a vacuum when the first valve is in the open position. In other aspects, the apparatus may comprise a second valve configured to control a flow of gas (e.g., pressurized gas) through the second conduit.

In certain aspects, the solvent may comprise water and/or ethanol. In several aspects, the probe is formed from polydimethylsiloxane (PDMS) and/or polytetrafluoroethylene (PTFE). In some aspects, the probe is disposable. In particular aspects, the probe may include a collection tip that is ejectable (e.g. capable of being ejected from the probe). In further aspects, the probe comprises a tracking device configured to track a location of the probe. In some aspects, the reservoir has a volume between 1 microliter and 500 microliters, between about 1 microliter and 100 microliters or between about 2 microliters and 50 microliters. In additional aspects, the reservoir has a volume between 5.0 microliters and 20 microliters.

In still further aspects, the apparatus may additionally comprise a control system configured to control: a solvent flow (e.g., flow of a fixed or discrete volume of solvent) from the chamber through the first conduit to the reservoir; a gas flow from the gas supply through the second conduit to the reservoir; and a sample flow from the reservoir through the third conduit to the mass spectrometer. In some aspects, the control system is configured to: control the solvent flow at a flow rate between 100 and 5000 microliters per minute (e.g., between 200 and 400 microliters per minute) for a period of time between 1 and 3 seconds; control the gas flow at a flow rate between 1 and 10 psig for a period of time between 10 and 15 seconds; and control the sample flow for a period of time between 10 and 15 seconds. For example, in some aspects, the control system comprises a trigger or button to initiate solvent flow. In further aspects, the control system comprises a pedal (i.e., that can be operated by foot action) to initiate solvent flow. A skilled artisan will recognize that the lengths of the first and/or second conduit may be adjusted to fit the particular use of the system. In yet further aspects, the control system is configured to control: a solvent flow (e.g., flow rate for a fixed period of time) from the chamber through the first conduit to the reservoir. In further aspects, an apparatus of the embodiments does not include a device for producing ultrasonic or vibrational energy (e.g., in sufficient amounts to disrupt tissues).

A further embodiment provided a method for assessing tissue samples from a subject comprising applying a solvent to a tissue site on the subject, collecting the applied solvent to obtain a liquid sample, and subjecting the sample to mass spectrometry analysis. In certain aspects, the solvent may be sterile. In some aspects, the solvent is pharmaceutically acceptable formulation. In specific aspects, the solvent is an aqueous solution. For example, the solvent may be sterile water or consist essentially of water. In other aspects, the solvent may comprise from about 1% to 5%, 10%, 15%, 20%, 25% or 30% of an alcohol. In some aspects, the solvent comprises 0.1% to 20% of an alcohol, 1% to 10% of an alcohol or 1% to 5% 1% to 10% of an alcohol (e.g., ethanol). In some cases, the alcohol may be ethanol.

In some aspects, applying the solvent to the tissue comprises applying a discrete volume of solvent to the tissue site. In some aspect, the solvent is applied in a single droplet. In a further aspect, the solvent is applied in a discrete number of droplets from 1 to 10. In some embodiments, the solvent is applied to the sample from the reservoir via a channel independent of the gas. In further embodiments, the solvent is applied to the sample under low pressure. For example, in some aspects, the solvent is applied by a mechanical pump such that solvent is applied to the tissue site (e.g., moved into a reservoir where it is in contact with the tissue site) with minimal force thereby exerting minimal pressure (and producing minimal damage) at a tissue site. The low pressure may be less than 100 psig, less than 90 psig, less than 80 psig, less than 70 psig, less than 60 psig, less than 50 psig, or less than 25 psig. In some embodiments, the low pressure is from about 0.1 psig to about 100 psig, from about 0.5 psig to about 50 psig, from about 0.5 psig to about 25 psig, or from about 0.1 psig to about 10 psig. In particular aspects, the discrete volume of solvent is between about 0.1 and 100 μL, or between about 1 and 50 μL. In further aspects, collecting the applied solvent is between 0.1 and 30 seconds after the applying step. In a specific aspect, collecting the applied solvent is between 1 and 10 seconds after the applying step (e.g., at least 1, 2, 4, 5, 6, 7, 8 or 9 seconds). In further aspects, a method of the embodiments does not involve application of ultrasonic or vibrational energy to a sample or tissue. In some aspects, the tissue site in an internal tissue site that is being surgically assessed.

In a further aspect, a method of the embodiments comprises applying a fixed or discrete volume of a solvent (e.g., using mechanical pump) to a tissue site through a solvent conduit. In some aspects, the fixed or discrete volume of a solvent is moved through a solvent conduit into a reservoir where it is in direct contact with a tissue site (e.g., for 0.5-5.0 seconds). In further aspects, collecting the applied solvent comprises applying a negative pressure to pull the sample into a collection conduit and/or applying a gas pressure to push the sample into a collection conduit. In some aspects, the solvent is applied through a solvent conduit that is separate from the collection conduit. In further aspects, wherein a gas pressure is applied to push the sample into the collection conduit the gas pressure is applied through a gas conduit that is separate from the solvent conduit and the collection conduit. In certain aspects, wherein a gas pressure is applied to push the sample into the collection conduit, the applied gas pressure of less than 100 psig. For example, the gas pressure can be less than 10 psig, such as 0.1 to 5 psig. In still further aspects, a method of the embodiments is defined as producing no detectable physical damage to the tissue being assessed.

In still further aspects, the method may additionally comprise collecting a plurality liquid samples from a plurality of tissue sites. In some cases, the device (e.g., the probe) used to collect the samples is washed between each sample collection. In other aspects, a device used to collect the samples includes a disposable collection tip (probe) that can be changed between each sample collection. In particular aspects, the collection tip may be ejectable (e.g. capable of being ejected from the device). In certain aspects, the plurality of tissue sites comprise 2, 3, 4, 5, 6, 7, 8, 9, 10 or more tissues sites in vivo. In another aspect, the plurality of tissue sites surround a section of tissue that has been surgically resected (e.g., ex vivo). In a specific aspect, the resected tissue is a tumor. In some aspects, the method may be defined as an intraoperative method.

A further embodiment provides a method of identifying a sampled tissue site and a method to communicate location of the site to the device (probe) operator. Identification of a sampled tissue site allows the operator to access the molecular information recorded at sampled tissue site at a time after sampling molecules collected from the tissue. At least three types of identification approaches are recognized. In the first approach, an exogenous material is attached to the sampled tissue site that identifies the sampled molecular information. In a second approach, the device (probe) is equipped with a tracking sensor/emitter that allows recording the location of the probe (device) and communication to an imaging device when the molecular information is sampled. In a third approach, the tissue region is modified so that the site may be easily identified after harvesting tissue molecules. In the first approach, materials that may be attached to the sampled tissue site include, for example, a suture, a surgical clip, a biocompatible polymer that adheres to the tissue, or an RFID chip that is attached to a magnetic bead that allows easy reading and removal. In the second approach type, the probe may contain an RF emitter that is part of a RF surgical tracking system, an ultrasound emitter or reflector that is part of an intra-operative US imaging system. In this second approach, when the operator initiates collection of tissue molecules, the tracking system records location of the probe in the associated imaging system (e.g., RF, US, CT, MRI) that may be in communication with the device. The operator may then identify any of the sampled tissue sites at a later time by referring to the recorded image(s) that can indicate the location of sampled sites to the operator. In the third approach, the tissue is modified. In this third approach, a laser source in communication with the probe may be used to ablate or coagulate a pattern into the tissue that identifies the sampled site. Any of these three approaches may be combined. For example, approach 1, 2 and 3 could be combined wherein an exogenous material is attached to the tissue site after harvesting tissue molecules and a laser patterns the exogenous tissue while an RF sensor records location of the harvest location and communicates to the imaging device.

In yet still further aspects, the mass spectrometry comprises ambient ionization MS. As disclosed herein a probe in contact with a tissue site can be in fluid communication with the MS via a conduit. In some aspects, conduit between the probe and tissue site is less than about 10 m, 8 m, 6 m or 4 m from MS. In further aspects, the conduit is between about 0.5, 1.0, 1.5, 2.0, 2.5, 3.0 and 4.0 m in length. In several aspects, subjecting the sample to mass spectrometry analysis may comprise determining a profile corresponding to the tissue site. In another aspect, the method may additionally comprise comparing the profile to a reference profile to identify tissue sites that include diseased tissue. In other aspects, the method also comprises resecting tissue sites that are identified to include diseased tissue. In some aspects, the method is performed using an apparatus in accordance with any of the embodiments and aspects described above.

In a further embodiment, the present disclosure may provide an ex vivo method for assessing tissue samples comprising obtaining a plurality of liquid samples from a plurality of tissue sites in a subject, subjecting the plurality of liquid samples to mass spectrometry to obtain a plurality of profiles corresponding to the tissue sites, and comparing the plurality of profiles to reference profiles to identify tissue sites that include diseased tissue. In certain aspects, the liquid samples are comprised in a solvent.

As used herein, “sample” or “liquid samples” can refer to extracts from tissues or other biological specimens (e.g., extracts comprising proteins and metabolites) obtained by contacting tissue or biological specimen with a solvent according to the embodiments. In some aspects, a sample can be an extract from a non-biological specimen, such as the surface on an object.

As used herein, “essentially free,” in terms of a specified component, is used herein to mean that none of the specified components has been purposefully formulated into a composition and/or is present only as a contaminant or in trace amounts. The total amount of the specified component resulting from any unintended contamination of a composition is therefore well below 0.01%. Some embodiments may use a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the terms “conduit” and “tube” are used interchangeably and refer to a structure that can be used to direct flow of a gas or liquid.

As used herein in the specification and claims, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating certain embodiments, are given by way of illustration only, since various changes and modifications within the spirit and scope of the disclosure will become apparent to those skilled in the art from this detailed description.

In certain aspects, the present disclosure provides methods and devices for minimally invasive molecular assessment of samples, such as tissue samples. In particular, aspects the methods can be used to assess sites of potential endometriosis, such as multiple tissue sites during an operation (or biopsy) of the tissue. This feature allows for accurate identification of diseased tissues (e.g., tissue sites retaining endometriosis) in “real-time” allowing surgeons to more accurately address only the diseased tissue relative to surrounding normal tissues. In particular aspects, the methods disclosed here can involve delivery of a fixed or discrete volume of solvent to a tissue site, followed by collection of a liquid sample from the site and analysis of the liquid sample by mass spectrometry. Importantly, rather than being applied in a high-pressure spray, solvent is applied as discrete droplets and at low pressure. These methods allow for accurate collection of samples from a distinct tissue site while avoiding damage to the tissue being assessed. The resulting mass spectrometry profile from collected samples allows for differentiation of diseased versus normal tissue sites. The method can be repeated at multiple sites of interest to very accurately map molecular changes (e.g., in a tissue). Importantly, the profiles of samples could be differentiated even without the use of an ionization source. Thus, while methods of the embodiments could be used in conjunction with an ionization source, the use of such a source is not required. These methodologies can allow assessment of plurality of tissue sites over a short range of time, thereby allowing for very accurate assessment of the boundaries of diseased versus normal tissues.

In some aspects, the materials (PDMS and PTFE) and solvent (e.g., water only solvents) used in the devices of the embodiments are biologically compatible, such that they can be used in surgery in for real-time analysis. Furthermore, because the devices can be very compact, it can be hand-held and used in used in minimally invasive surgical procedures, or non-surgical procedures.

In some aspects, the present disclosure provides devices of extended length and increased compactness for delivery of fixed or discrete volumes of solvents to tissues for use in minimally invasive surgeries. In some aspects, these methods can be encapsulated in a variety of form factors such as a conduit, ranging from 0.5 mm to 10.0 mm inner diameter (e.g., with an inner diameter of between about 1.0 and 5.0; 1.0 and 10.0; 2.0 and 8.0; or 5.0 and 10.0 mm). In some aspects, the site of delivery of a fixed or discrete volume of solvent, followed by collection of a liquid sample may be inside the body, such as a surgical site. In some aspects, two smaller conduits may be inserted into a third, larger, conduit to create a multi-lumen catheter. For example, the multi-lumen catheter can have 2, 3, 4, 5, 6 or more luminal spaces with each having an internal diameter of, e.g., 0.05 to 5.0 mm; 0.1 to 5.0 mm; 0.25 to 3.0 mm; or 0.5 mm to 10.0 mm. The multi-lumen catheter may be attached to a mass spectrometry device for analysis of sample tissues inside the body during surgery, while avoiding unnecessary damage to surrounding tissues.

In some aspects, the device may be used through cannulas or catheters in minimally invasive surgical or endoscopy procedures, or may be used in non-surgical procedures through needle guides or biopsy guides. In some aspects, the present disclosure can be integrated into a robotic surgical system allowing several regions of the human body cavity to be quickly sampled and analyzed. In some aspects, the device be used to analyze tissues using a database of molecular signatures and machine learning algorithms, allowing diagnosis in real time for each sampled region. The present disclosure may be used in a wide variety of oncological and other surgical interventions, such as endometriosis, for which real time characterization and diagnosis of tissues are needed.

In some aspects, the present disclosure provides an attachment to the probe, for fine manipulation of the probe during minimally or non-invasive procedures. For example, the attachment to the probe may be a fin. In some aspects, such a fin may be composed of the same material as the probe. In some cases, the fin is made of PDMS. A fin can, in some aspects, be formed by an injection molding process or it may be 3D printed. In some aspects, the present disclosure includes a device for grasping the probe, external to the probe, in order to manipulate the probe during laparoscopic procedures. The grasping device may be used to hold, rotate, or move the probe, or may grasp the fin attached to the probe, in order to move or rotate the probe.

In some aspects, the present disclosure maintains a reservoir using a multi-lumen catheter with recessed ports for depositing water and nitrogen gas during laparoscopic surgical procedures. A multi-lumen catheter may be formed, for example, using a multi-lumen extrusion as is well known in the art. These catheters may be utilized in any cannula. The most commonly used cannulas are of 5 mm and 10 mm diameters, and are typically used for laparoscopic surgeries.

In some aspects, the present disclosure provides tools, devices and methods for manipulation of the probe during endoscopy. For example, multi-lumen tubing may be used with an external vacuum source in order to attach the probe to the tissue surface while analyzing.

In some aspects, the present disclosure provides a shutter system that occludes the orifice of the minimally invasive surgical device. In some aspects, this shutter system may be a catheter balloon that is integrated within the device or added separately to the device. The shutter, or balloon, may close the probe tip, preventing unwanted biological material from entering the device, including the lumens and tubing, upon insertion of the catheter into the patient. The shutter or balloon may disallow endogenous biological fluids from entering the mass spectrometer after analysis has been initiated, thus preventing contamination of the results. Finally, closing of the shutter or balloon may prevent excess nitrogen gas and water from entering the body. Inclusion of lengthened probes for minimally invasive surgeries and occlusion technologies for the tips of the probes may mitigate the unpredictable and often tumultuous nature of internal organ movement and organ systems during surgery which could affect signal acquisition. Balloons technologies could also be used in other region of the device instead or in addition to the pinch valves to control solvent and gas motions through the tubes.

In some aspects, the present disclosure may be used with robotic manipulation. In some aspects, the technologies of the present disclosure may integrate in modern surgical theaters through an accessory port, or via a robotic arm. These devices may be integrated into robotic systems such as the Intuitive Surgical da Vinci robotic surgical system. A device of the present disclosure may have its own dedicated arm in a robotic system, or be handled by robotic graspers by incorporating a “fin” onto the probe. Smaller and larger diameters can also be used to be coupled to any existing catheters, cannulas and also needle/biopsy guides.

In some aspects, a tracking probe can be integrated with this device in order to display and record where the tissue sample has been analyzed to better assist the surgeon in localizing the sampling points both intraoperatively or otherwise. For example, during intraoperative ultrasound, an ultrasound emitter on the device may be utilized to display the probe when sampling. The probe may be integrated with a tracking device based on radio frequency technology, such as the Biosense Webster Carto system. In that case, the probe may display the device/sampling location on any of a variety of imaging modalities, such as intraoperative UltraSound (US)/Computed Tomography (CT)/Magnetic Resonance Imaging (MRI)/Optical Coherence Tomography (OCT). Additionally, fluorescent imaging and molecular dyes may be used to track the analyzed areas and charted to provide 2-dimensional or 3-dimensional spatial imaging. More simply, the probe tip may be coated with a surgical dye which is then stamped on the tissue to track the region analyzed. Yet another tracking approach is to integrate an RF emitter into the probe so that the spatial location may be tracked.

In some aspects, the probe of the present disclosure may be used to assist surgeons and medical professionals during minimally invasive surgical interventions by providing comprehensive and definitive diagnostic molecular information in vivo and in real time, without necessarily causing damage or alteration to the patient's native living tissues. The handheld MasSpec Pen has demonstrated a capacity to do this during non-laparoscopic/endoscopic surgical procedures (U.S. patent application Ser. No. 15/692,167 incorporated herein by reference, in its entirety). Similarly to the handheld MasSpec Pen, the present disclosure is suitable for ex vivo analysis of tissues (fresh, frozen, sections, biopsies) or other clinical specimens that might be examined by a pathologist, and may be used for chemical analysis of any given sample for which direct analysis is desired in confined and spatially limited domains (animals, plants, explosives, drugs, etc). A variety of tissue types may be analyzed as well, including but not limited to, breast, kidney, lymph node, thyroid, ovary, pancreatic and brain tissues.

In some aspects, the probe of the present disclosure may be used in conjunction with surgical instruments for the treatment of a disease. A variety of surgical instruments may be used to excise or ablate cells or tissues, including, but not limited to, laser ablation tools, tools for cauterization or electrocauterization, or tools for the manual dissection of tissue such as a scalpel.

Thus, many regions of the human body cavity can be quickly sampled during surgery, and analyzed (e.g., by using a database of molecular signatures and machine learning algorithms). Therefore, the diagnostic results may be provided in real time for each sampled region. Exemplary devices for use in these methods are detailed below.

### Exemplary Features of a Device of the Embodiments

### Shutter Systems

In some aspects a device of the embodiments further comprises a shutter system that can occlude the orifice, and creates a separation between the reservoir and the tissue. For example, the shutter system can activate after the droplet rests for 3 seconds and before the droplet is transported to the mass spectrometer. One reason for this is to ensure no biological material reach the mass spectrometer and cause damage to the instrument. The shutter can be an iris diaphragm, a mechanical closure, gate, or tapenade. An additional design for the shutter is a balloon mechanism, which seals the exterior of the device from the tissue. The balloon can be positions on the distal end of the conduit, e.g., perpendicular to the pen or probe. When activated, the balloon expands and fills up the reservoir towards the direction of the tissue. This accomplishes at least 3 things: first it gently lifts the pen tip off of the tissue using the inflated balloon, insuring that there is no damage to the tissue. This is to ensure that the probe remains nondestructive and biocompatible in case the analyzed tissue is determined to be ‘normal’. Secondly, it seals the solvent droplet that is inside the reservoir and prevents leakage or absorbance of lipids after the sampling window. Thirdly, it creates a seal at the end of the conduit, which will allow for more effective transfer of the droplet to the mass spectrometer.

### Catheter Systems

In some cases, where a probe is incorporated into a laparoscopic/endoscopic device a reservoir includes using a multi-lumen catheter, e.g., with recessed ports for depositing water and nitrogen gas. The reservoir also retains the water during the extraction period. A multi-lumen catheter can be formed for example using a multi-lumen extrusion as is well known in the art. It has been demonstrated that these catheters can be utilized in any cannula, most commonly 5 mm and 10 mm diameters, for laparoscopic surgeries. This technology is compatible with robotic manipulation such as the Intuitive Surgical da Vinci robotic surgical system. The Laparoscopic/Endoscopic probes will easily integrate in current surgical theaters through an accessory port or via a robotic arm. Smaller and larger diameters can also be used to be coupled to any existing catheters, cannulas and also needle/biopsy guides.

### Valve Systems

In further aspects, a probe system of the embodiments can incorporate additional valves. For example, micro-solenoid valves can be located at each conduit, e.g., at the distal end of the sampling probe. These will be individually controlled by an arduino, microcontroller, or signal. In some cases the value operation is automated. In other cases it can be manually controlled. In some aspects, valves are positioned in the inner wall of the solvent conduit sealing the conduits. Thus, by using such values, only two or even one conduit can be used in the sampling operation. For example, a delivering solvent conduit and a return conduit to transfer the droplet to the mass spectrometer. Additional micro-solenoids could be implanted to have more control. For example, three or four micro-solenoids can be into the probes of the embodiments.

### Further Surgical System Features

In some aspects, medical devices require passage to areas of the body that are difficult to maintain manual control. One solution is to use endoscopic catheters, but these are often less precise when compared to handheld devices. Further control can be attained using robotic tools that can function nearly to the same extent, and sometimes better than physicians equipped with a traditional scalpel. A further feature of the Laparoscopic/Endoscopic probes of the embodiments is a ‘fin’ that can be grasped by forceps, robotic tools, or laparoscopic graspers. This will allow the probe to be used in a variety of modalities without sacrificing resolution or sensitivity. In some aspects, the fin itself is a gradual sloped protrusion from the exterior of the conduit running parallel to said conduit. It is textured to provide extra traction for the grasping mechanism.

In further aspects, a tracking probe can be integrated with this device in order to display and record where the tissue sample has been analyzed to better assist the surgeon in localizing the sampling points both intraoperatively or otherwise. For intraoperative ultrasound, an ultrasound emitter on the device may be utilized to display the probe when sampling. Alternatively, the probe can be integrated with a tracking device based on radio frequency technology, such as the e.g., Biosense Webster Carto system. With this approach, the probe displays the device/sampling location on any various imaging modalities like intraoperative UltraSound (US)/Computed Tomography (CT)/Magnetic Resonance Imaging (MRI)/Optical Coherence Tomography (OCT).

In some further aspects, tissue sites that are assessed by a probe of the embodiments can be marked. For example, a dye that is up-taken by endometrial cells and normal cells, which will mark where the probe has been placed. In some aspects, a chemical dye can be delivered using an additional conduit in the catheter or by using a multilumen catheter. An alternative delivery of a tracking dye is to dissolve it in the solvent that we use to analyze the tissue. For instance, one advantage of using a dye within the solvent is that it will directly correlate with where the tissue sample was taken, instead of the peripheral region. Of course in this aspects, the chemical dye would be present in the mass spectra and would have to be distinguished from biomolecules in a sample. In some aspects, it may useful to make the dye visible (e.g., in white operating room light). In other aspects, the dye may be a fluorescent dye. In yet a further aspect, the pen tip can be coated with a surgical dye, which is then stamped on the tissue to track the region analyzed. Likewise, as discussed above, a tracking approach can be used to virtually map the tissues sites analyzed. For instance, a RF emitter can be integrated into a probe so that the spatial location may be tracked. Thus, in some aspects, dyes (or probe tracking) can be used to track analyzed areas of tissues. In some aspects, tissues analyzed can be charted to provide 2 dimensional and 3 dimensional spatial imaging.

In further aspects, a probe system can include a filter. For example a filter can prevent biological tissue from going into the conduits. For example, a filter mesh system can be incorporated within the device to prevent smaller bodies of tissue, protein aggregates, or coagulated cell clusters from entering. This mesh could be placed at the opening and have contact with the tissue, or be positioned higher up within the probe, such that no tissue contact occurs. In some aspects, such a filter mesh comprises average aperture sizes of less than about 1.0, 0.5, 0.25 or 0.1 mm. Since solid matter can damage a mass spectrometer, such a filter system can increase instrument lifespan without negatively effecting signal detected.

In still further aspects, an endoscopic/laparoscopic probe of the embodiments is integrated with a microcontroller, user interface, and/or associated hardware that will operate with appropriate software.

In some further cases, a light, such as a LED will be incorporated to provide visual feed back to the user, for example, to indicate that the probe is ready for sampling, in the process of doing so, or needs to be replaced/repaired. Acoustic feedback can also be used, for instance, to let the user know what step of the process the device is in (e.g., since physical cues may be unavailable laparoscopically). A user interface system can also be integrated with the device, such as in a foot pedal and buttons on the housing of the probe.

### Assay Methodologies

In some aspects, the present disclosure provides methods of determining the presence of diseased tissue (e.g., tumor tissue) or detecting a molecular signature of a biological specimen by identifying specific patterns of a mass spectrometry profile. Biological specimens for analysis can be from animals, plants or any material (living or non-living) that has been in contact with biological molecules or organisms. A biological specimen can be samples in vivo (e.g. during surgery) or ex vivo.

A profile obtained by the methods of the embodiments can correspond to, for example, proteins, metabolites, or lipids from analyzed biological specimens or tissue sites. These patterns may be determined by measuring the presence of specific ions using mass spectrometry. Some non-limiting examples of ionizations methods that can be coupled to this device include chemical ionization, laser ionization, atmospheric-pressure chemical ionization, electron ionization, fast atom bombardment, electrospray ionization, thermal ionization. Additional ionization methods include inductively coupled plasma sources, photoionization, glow discharge, field desorption, thermospray, desorption/ionization on silicon, direct analysis in real time, secondary ion mass spectrometry, spark ionization, and thermal ionization.

In particular, the present methods may be applied or coupled to an ambient ionization source or method for obtaining the mass spectral data such as extraction ambient ionization source. Extraction ambient ionization sources are methods with, in this case, liquid extraction processes dynamically followed by ionization. Some non-limiting examples of extraction ambient ionization sources include air flow-assisted desorption electrospray ionization (AFADESI), direct analysis in real time (DART), desorption electrospray ionization (DESI), desorption ionization by charge exchange (DICE), electrode-assisted desorption electrospray ionization (EADESI), electrospray laser desorption ionization (ELDI), electrostatic spray ionization (ESTASI), Jet desorption electrospray ionization (JeDI), laser assisted desorption electrospray ionization (LADESI), laser desorption electrospray ionization (LDESI), matrix-assisted laser desorption electrospray ionization (MALDESI), nanospray desorption electrospray ionization (nano-DESI), or transmission mode desorption electrospray ionization (TM-DESI).

As with many mass spectrometry methods, ionization efficiency can be optimized by modifying the collection or solvent conditions such as the solvent components, the pH, the gas flow rates, the applied voltage, and other aspects which affect ionization of the sample solution. In particular, the present methods contemplate the use of a solvent or solution which is compatible with human issue. Some non-limiting examples of solvent which may be used as the ionization solvent include water, ethanol, methanol, acetonitrile, dimethylformamide, an acid, or a mixture thereof. In some embodiments, the method contemplates a mixture of acetonitrile and dimethylformamide. The amounts of acetonitrile and dimethylformamide may be varied to enhance the extraction of the analytes from the sample as well as increase the ionization and volatility of the sample. In some embodiments, the composition contains from about 5:1 (v/v) dimethylformamide:acetonitrile to about 1:5 (v/v) dimethylformamide:acetonitrile such as 1:1 (v/v) dimethylformamide:acetonitrile. However, in an example embodiment, the solvent for use according to the embodiments is a pharmaceutically acceptable solvent, such as sterile water or a buffered aqueous solution.

### EXAMPLES

The following examples are included to demonstrate example embodiments of the present disclosure. Those of skill in the art should, in light of the present disclosure, appreciate that many changes can be made in the example embodiments and still obtain a like or similar result without departing from the spirit and scope of the present disclosure.

### Example 1—Molecular Analysis of Endometriosis Using a Laparoscopic MasSpec Pen to Aid in Surgical Resection

Endometriosis typically entails uncontrolled growth of endometrial tissue outside the uterus. Endometriosis affects approximately 10% women in their reproductive years. Symptoms can include pelvic pain, abdominal distortion, and subfertility. Currently, causes and pathogenesis of endometriosis is unclear and no proposed biomarkers have proven capable of disease diagnosis. Typically, the best treatment option is laparoscopic removal of endometriosis lesions, although ~50% of patients see recurrence of lesions within five years. Current endometriosis diagnosis procedure typically involves a patient with non-specific symptoms being subjected to a pelvic exam, ultrasound, and/or MRI followed by exploratory surgery and formalin-fixed paraffin-embedded (FFPE) section analysis. Diagnosis of endometriosis may be confirmed by observation of histological features such as hemosiderin, endometrial stroma, and/or endometrial glands. Treatment may include hormone treatment and/or resection surgeries. However, incomplete resection can lead to disease recurrence but simultaneously special attention must be paid to conserve healthy tissue.

The gross anatomy of endometriosis complicates resection further. Endometriosis lesions can have a variety of appearances, some of which can be difficult to identify by an untrained eye. Endometriosis can present as “invisible” microscopic lesions that are difficult for even expert surgeons to remove. Thus, methods for in vivo detection of endometriosis lesions are desirable and may provide more confident diagnosis of endometriosis during surgery, allowing more complete resection during exploratory surgery while avoiding healthy adjacent structures.

Mass spectral characterization features of endometriosis include lower quantities of lactate, gluconate, and arachidonic acid mass peaks and increased quantities of ascorbate, oleic acid, and glycerophosphoserine mass peaks. Small endometriosis lesions, 16 out of 20 misclassified normal samples were from soft tissue, and 34 of the 42 endometriosis lesions embedded in soft tissue, were reflective of oversampling of the endometriosis lesions with the 2.7 mm reservoir tip.

Further, to transition the use of the MasSpec Pen into a laparoscopic environment, the pen underwent further modifications. The initial size of the MasSpec Pen tip was 12 mm, the device was manipulated by hand, and the operation was performed in a primarily N₂ environment. The pen was modified for laparoscopic use by reducing the size of the tip to fit in laparoscopic trocars (~8 mm), the device was modified for manipulation using laparoscopic instruments, and was designed for operation in a primarily CO₂ environment The modified MasSpec Pen was tested laparoscopically in vivo. The modified pen may be inserted into a surgical trocar as a drop-in probe, may be maneuvered with multiple designs of surgical forceps, and laparoscopic use allows for visualization of the location of analysis. The difference in mass spectral data in an N₂ environment vs. a CO₂ environment were investigated.

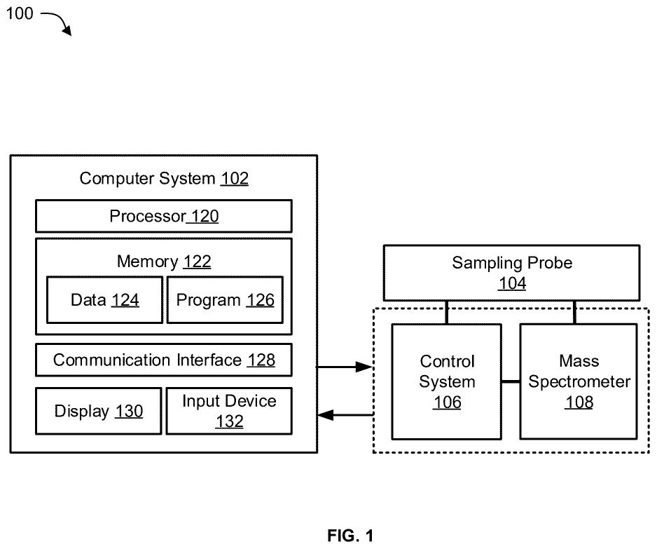

FIG. 1 is a schematic diagram of an example system 100. As shown in FIG. 1, the example system 100 includes a computer system 102, a sampling probe 104, a control system 106, and a mass spectrometer 108. In some implementations, the example system 100 may be used for qualitatively and quantitatively tissue analysis and identification of endometriosis. In some examples, the example system 100 may include additional or different components, and the components may be arranged as shown or in another manner.

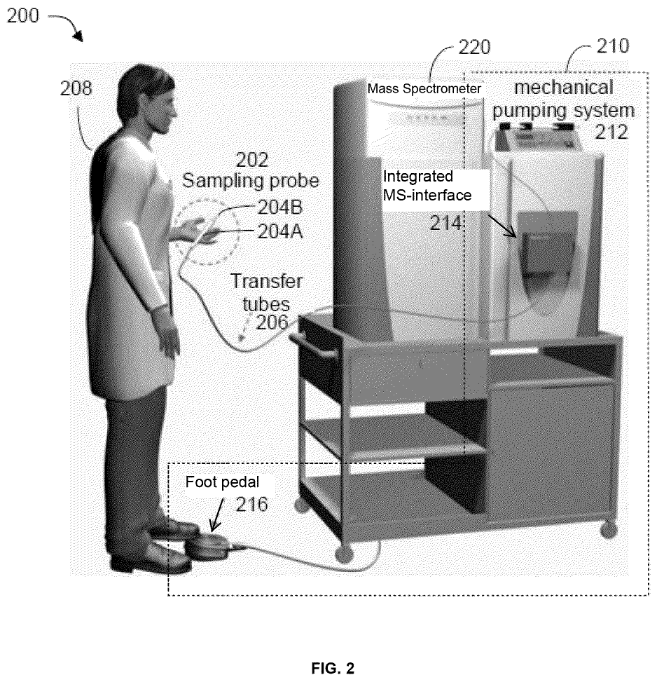

In the example shown in FIG. 1, the computer system 102 includes a processor 120, memory 122, a communication interface 128, a display device 130, and an input device 132. In some implementations, the computer system 102 may include additional components, such as, for example, input/output controllers, communication links, power, etc. In some implementations, the computer system 102 may be configured to control operational parameters of and to receive data from the control system 106, and the mass spectrometer 108. The computer system 102 can be used to control the control system 106 to deliver a liquid solvent to the sampling probe, and to obtain a liquid sample by extracting a liquid solvent carrying suspended cells and/or extracted molecules from the cells. The computer system 102 may be used to operate the mass spectrometer 108 to perform a tissue analysis on the liquid sample to obtain mass spectrometry data. In some implementations, the computer system 102 may be used to implement one or more aspects of the systems and processes described with respect to FIGS. 2, 3, and 4, or to perform other types of operations. In some implementations, the computer system 102 includes a separate control unit associated with and providing specific control functions to the control system 106.

In some implementations, the computer system 102 may include a single computing device, or multiple computers that operate in proximity to the rest of the example system 100 (e.g., the control system 106 and the mass spectrometer 108). In some implementations, the computer system 102 may communication with the rest of the example system 100 via the communication interface 128 through a communication network, e.g., a local area network (LAN), a wide area network (WAN), an inter-network (e.g., the Internet), a network comprising a satellite link, and peer-to-peer networks (e.g., ad hoc peer-to-peer networks).

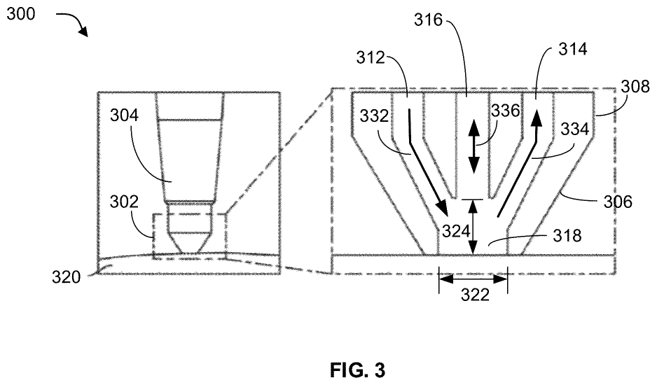

In some implementations, the sampling probe 104 may be configured to provide fluidic communication with the control system 106 and the mass spectrometer 108 via transfer tubes. In some aspects of operation, the sampling probe 104 receives liquid solvent from the control system 106, guides the liquid solvent to a tissue site with possible endometriosis tissue, obtains a liquid sample by extracting at least a portion of the liquid solvent with the suspended cells and/or extracted molecules, and guides the liquid sample to the mass spectrometer 108. In some implementations, the sampling probe 104 may include a probe tip, which may include multiple internal liquid/gas channels and an internal reservoir, e.g., the channels 312, 314, 316 and the internal reservoir 318 as shown in FIG. 3 or in another manner. In some implementations, the sampling probe 104 may be composed of materials, such as synthetic polymers that are biologically compatible and resistant to chemical compounds under measurement. In some examples, the sampling probe 104 may be implemented as the sampling probes 202, 300 as shown in FIGS. 2-3 or in another manner.

The example control system 106 controls the movement of fluid in the system 100. In some implementations, the control system 106 includes a mechanical pumping system and one or more mechanical valves. In some instances, the mechanical pumping system includes a mechanical pump that is controlled by the computer system 102, which may provide high-precision, microfluidic dispensation of the liquid solvent to the internal reservoir of the sampling probe 104. In some implementations, the liquid solvent may be polar or non-polar, which may include sterile water, a type of alcohol, an internal standard, or a combination. In some implementations, the control system 106 may be implemented as the control system 210 as shown in FIG. 2 or in another manner. In some instances, a control unit of the control system 106 (e.g., the integrated MS interface 214) may be configured to trigger and control a sampling process by controlling the mechanical pumping system and the one or more mechanical valves. Simultaneously, the control unit of the control system 106 may be configured to trigger a data collection process by the mass spectrometer 108.

In some implementations, the example system 100 may include an ionization system. In certain instances, the liquid sample may be ionized and transferred to the mass spectrometer 108. In some implementations, the mass spectrometer 108 may include a mass selector and a mass analyzer, which are configured to separate and identify molecules according to their mass-to-charge (m/z) ratio. In some implementations, the mass spectrometer 108 may output a set of mass spectra (e.g., relative abundance of charged molecules vs. m/z ratio plot) to the computer system 102, which may be stored in the memory 122, analyzed by running a program 126 and results may be further displayed on the display 130. In some implementations, the mass spectrometer 108 may be implemented as the mass spectrometer 220 as shown in FIG. 2 or in different manner.

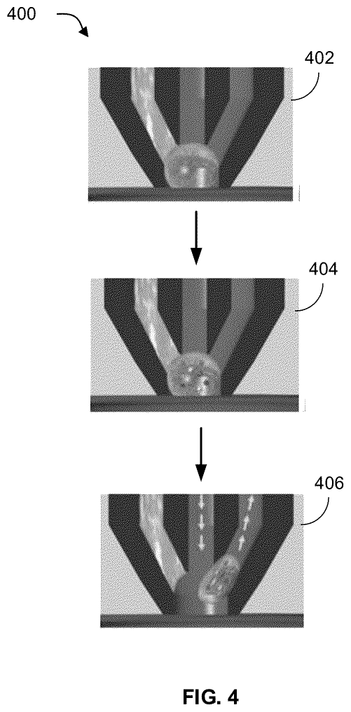

In some implementations, some of the processes and logic flows described in this specification can be automatically performed by one or more programmable processors, e.g. processor 120, executing one or more computer programs to perform actions by operating on input data and generating output. For example, the processor 120 can run the programs 126 by executing or interpreting scripts, functions, executables, or other modules contained in the programs 126. In some implementations, the processor 120 may perform one or more of the operations described, for example, with respect to FIG. 4.

In some implementations, the processor 120 can include various kinds of apparatus, devices, and machines for processing data, including, by way of example, a programmable data processor, a system on a chip, or multiple ones, or combinations, of the foregoing. In certain instances, the processor 120 may include special purpose logic circuitry, e.g., an Arduino board, an FPGA (field programmable gate array), an ASIC (application specific integrated circuit), or a Graphics Processing Unit (GPU) for running the deep learning algorithms. In some instances, the processor 120 may include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. In some examples, the processor 120 may include, by way of example, both general and special purpose microprocessors, and processors of any kind of digital computer.

In some implementations, the processor 120 may include both general and special purpose microprocessors, and processors of any kind of quantum or classic computer. Generally, a processor 120 receives instructions and data from a read-only memory or a random-access memory or both, e.g. memory 122. In some implementations, the memory 122 may include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and CD ROM and DVD-ROM disks. In some cases, the processor 120 and the memory 122 can be supplemented by, or incorporated in, special purpose logic circuitry.

In some implementations, the data 124 stored in the memory 122 may include, operational parameters, a standard reference database and output data. In some implementations, the standard reference database may include a mass spectral reference library. In some instances, the output data may include mass spectrometry data and statistical analysis results. In some implementations, the programs 126 can include software applications, scripts, programs, functions, executables, or other modules that are interpreted or executed by the processor 120. In some instances, the programs 126 may include machine-readable instructions for delivering the liquid solvent to the sampling probe, collecting the liquid sample from the sampling probe, and subjecting the liquid sample for mass spectrometry analysis. In some instances, the programs 126 may obtain input data from the memory 122, from another local source, or from one or more remote sources (e.g., via a communication link). In some instances, the programs 126 may generate output data and store the output data in the memory 122, in another local medium, or in one or more remote devices (e.g., by sending the output data via the communication network 106). In some examples, the programs 126 (also known as, software, software applications, scripts, or codes) can be written in any form of programming language, including compiled or interpreted languages, declarative or procedural languages. In some implementations, the programs 126 can be deployed to be executed on the computer system 102.

In some implementations, the communication interface 128 may be connected to a communication network, which may include any type of communication channel, connector, data communication network, or other link. In some instances, the communication interface 128 may provide communication with other systems or devices. In some instances, the communication interface 128 may include a wireless communication interface that provides wireless communication under various wireless protocols, such as, for example, Bluetooth®, Wi-Fi, Near Field Communication (NFC), GSM voice calls, SMS, EMS, or MMS messaging, wireless standards (e.g., CDMA, TDMA, PDC, WCDMA, CDMA2000, GPRS) among others. In some examples, such communication may occur, for example, through a radio-frequency transceiver or another type of component. In some instances, the communication interface 128 may include a wired communication interface (e.g., USB, Ethernet) that can be connected to one or more input/output devices, such as, for example, a keyboard, a pointing device, a scanner, or a networking device such as a switch or router, for example, through a network adapter.

In some implementations, the communication interface 128 can be coupled to input devices and output devices (e.g., the display device 130, the input device 132, or other devices) and to one or more communication links. In the example shown, the display device 130 is a computer monitor for displaying information to the user or another type of display device. In some implementations, the input device 132 is a keyboard, a pointing device (e.g., a mouse, a trackball, a tablet, and a touch sensitive screen), or another type of input device, by which the user can provide input to the computer system 102. In some examples, the computer system 102 may include other types of input devices, output devices, or both (e.g., mouse, touchpad, touchscreen, microphone, motion sensors, etc.). The input devices and output devices can receive and transmit data in analog or digital form over communication links such as a wired link (e.g., USB, etc.), a wireless link (e.g., Bluetooth®, NFC, infrared, radio frequency, or others), or another type of link.

In some implementations, other kinds of devices may be provided for interaction with a user as well; for example, feedback provided to the user can be any form of sensory feedback, e.g., visual feedback, auditory feedback, or tactile feedback; and input from the user can be received in any form, including acoustic, speech, or tactile input. For example, the sampling probe 104 may contain a control element (e.g., button, foot pedal, etc.) which may be used as a controller to initiate, interrupt, restart, or terminate a detection process (e.g., the foot pedal 216 as shown in FIG. 2). In some instances, a graphic user interface (GUI) may be used to provide interactions between a user and the example system 100. In certain instances, the GUI may be communicably coupled to the computer system 102. For example, when the control system 106 is activated (e.g., by pushing on the foot pedal 216 in FIG. 2), the GUI can simultaneously initiate a sampling process on the control system 106 and a tissue analysis process on the mass spectrometer 108. For example, when the tissue analysis process is completed, the GUI can output and display a report with analysis results.

FIG. 2 is a schematic diagram showing aspects of an example system 200. In the example shown in FIG. 2, the system 200 includes a sampling probe 202, a control system 210, and a mass spectrometer 220. As shown in FIG. 2, the sampling probe 202 is coupled between the control system 210 and the mass spectrometer 220 through transfer tubes 206. In some examples, the system 200 may include additional or different components, and the components may be arranged as shown or in another manner.

In the example shown in FIG. 2, the sampling probe 202 includes a housing 204A and a probe tip 204B. In some implementations, the housing 204A may provide a grip for being used as a hand-held sampling probe that can be operated by a user 208. In some implementations, the housing 204A may include a control element, e.g., a trigger or button. For example, the control element may be used to control the liquid solvent transferring through the sampling probe 202. In some instances, the control element may be separated from the housing 204A, e.g., configured as the foot pedal 216 of the control system 210. For another example, the control element may be coupled to a mechanism which may be used to eject the probe tip 204B. In some implementations, the sampling probe 202 may be composed of materials, such as synthetic polymers that are biologically compatible and resistant to chemical compounds under measurement. For example, the materials for the sampling probe 202 may be compatible with a variety of liquid solvent (e.g., polar or non-polar) that is used for extracting and carrying a liquid sample to the mass spectrometer 220. In some examples, the synthetic polymers that may be used for fabricating the sampling probe 202 may include Polydimethylsiloxane (PDMS), or Polytetrafluoroethylene (PTFE). In some implementations, the probe tip 204B may use the same material as the housing 204A, different materials or different compositions.

In some implementations, the sampling probe 202 may be manufactured using a 3D printing process, a machining process, or another process. In some implementations, the housing 204A of the sampling probe 202 may include two internal channels which are fluidically coupled with the transfer tubes 206 and respective channels in the probe tip 204B. In some implementations, the transfer tubes 206 supplies a liquid solvent to the probe tip 204B and transfers a liquid sample which includes at least a portion of the liquid solvent with suspended cells and/or extracted molecules from the probe tip 204B to the mass spectrometer 220. The sampling probe 202 may also include a gas channel (e.g., an open port that receives air from the surrounding atmosphere) that allows liquid to be flushed from the sampling probe 202, for example, between uses or at other instances.

In some implementations, the probe tip 204B may be detachable from the housing 204A, which can be disposed and replaced if contaminated, e.g., after a certain number (e.g., one or more) of regular uses or when switching between different samples. In some cases, the probe tip 204B may include internal channels that are fluidically coupled to the respective channels in the housing 204A and further to the transfer tubes 206. In some implementations, the probe tip 204B may be integrated with the housing 204A as a monolithic structure. In some implementations, the probe tip 204B may be implemented as the probe tip 302 as shown in FIG. 3 or in another manner.

In some implementations, the control system 210 may include a solvent container and a mechanical pumping system 212. In some instances, the mechanical pumping system 212 may contain one or more mechanical pumps. In some instances, the one or more mechanical pumps may be programable. In certain examples, the one or more mechanical pumps may be controlled by a computer system, e.g., the computer system 102 in FIG. 1. In some implementations, a mechanical pump may include a syringe pump, a peristatic pump or another type of pump, which can provide high-precision, microfluidic dispensation of the liquid solvent from the solvent container to the probe tip 204B, e.g., the internal reservoir 318 of the probe tip 302 as shown in FIG. 3. In some implementations, each of the one or more mechanical pumps may be equipped with separate solvent containers containing different types of liquid solvents. In some instances, different types of liquid solvents may be selected or mixed. In the example shown in FIG. 2, the liquid solvent in a container (e.g., syringe) is delivered to the sampling probe 202 through the transfer tubes 206. In some implementations, the control system 210 can supply a controlled volume of liquid solvent to the sampling probe 202 at a controlled flow rate according to the design of the example system, e.g., the length of the transfer tubes 206, the volume of the internal reservoir 318 and the diameter of the liquid channels 312, 314 as shown in FIG. 3.

In the example shown in FIG. 2, the control system 210 may further includes one or more valves on the transfer tubes 206. In some implementations, each of the one or more valves is configured to control a fluidic flow (e.g., start or stop a fluidic flow) in the transfer tubes 206. In some implementations, each of the one or more valves may be mechanically activated and electrically controlled by a computer system, e.g., the computer system 102 as shown in FIG. 1. In some examples, the one or more valves may include a pinch valve, a squeeze valve, other type of valve, or a combination. In some instances, the valve on the transfer tubes 206 is a high-speed actuated pinch valve for controlling aspiration and extraction of the liquid sample to the mass spectrometer 220. In some instances, the control system 210 is communicably coupled with an integrated mass spectrometer (MS) interface 214. In some instances, the integrated MS interface 214 may include an Arduino board to control motions of the mechanical pumping system 212 and the one or more valves. As shown, the integrated MS interface 214 can be activated by pushing the foot pedal 216 and deactivated by releasing the foot pedal 216. In some instances, when activated, the integrated MS interface 214 may also initiate a data collection process performed by the mass spectrometer 220.

In some implementations, the transfer tubes 206 may have an inner diameter of 0.8 mm and may be made of biocompatible synthetic polymers, e.g., polytetrafluoroethylene (PTFE). In some implementations, the transfer tubes 206 may have a length of one or more meters (e.g., approximately 1.5 m) to allow free handheld use of the sampling probe 202 by an operator without geometrical or spatial constraints.

In some implementations, the mass spectrometer 220 may include a mass selector and a mass analyzer. In some implementations, the mass selector may separate fragment ions by dissociating the molecules in the liquid sample according to their mass-to-charge (m/z) ratio based on dynamics of charged particles in electric and magnetic field in vacuum. The mass selector may include a set of magnets providing a magnetic field, which the fragment ions travel through. In some instances, the mass selector may use the magnetic field to alter the path of the fragment ions so that they can be separated according to their charges and masses. The mass analyzer may include detectors to identify fragment ions. In some examples, the mass spectrometer 220 can output a set of mass spectra (or mass spectrometry data in another format) for data analysis.

In some aspects of operation, the example system 200 may include an ionization system to receive and ionize the liquid sample. In some examples, the ionized liquid sample may include the fragment ions of the dissociated molecules from the liquid solvent, the suspended cells, and/or the extracted molecules from the cells. In some implementations, the ionized liquid sample may be transfer out from the ionization system to the mass spectrometer 220. In some aspects of operation, the ionized liquid sample may be filtered, captured and analyzed by the mass spectrometer 220. In some implementations, prior to reaching the mass spectrometer, the ionized liquid sample may be collected and delivered to an ion optic system. In some instances, the ion optic system may be configured to filter neutral species in the ionized liquid sample, to allow ions passing through, and to eliminate contamination of the mass spectrometer 220.

FIG. 3 is a schematic diagram showing aspects of a sampling probe 300 in an example system. As shown in FIG. 3, the sampling probe 300 includes a probe tip 302 and a housing 304. The example probe tip 302 includes one mandrel end 306 in a tapered cylindrical shape which is used for contacting a sample surface 320, and one cylindrical end 308 which is used to engage with a receiving end of the housing 304. In some implementations, the cylindrical end 308 may make an air-tight seal with the receiving end of the housing 304. In some examples, the probe tip 302 may include additional or different components, and the components may be arranged as shown or in another manner.

As shown in a cross-sectional view of the probe tip 302 in FIG. 3, the probe tip 302 includes three distinct internal channels (e.g., conduits), including a liquid supply channel 312, a liquid extraction channel 314, and a gas channel 316. In some implementations, the three internal channels 312, 314, 316 are aligned with respective internal channels (not shown) in the receiving end of the housing 304 to provide fluidic communication with transfer tubes. In some instances, the transfer tubes may be implemented as the transfer tubes 206 as shown in FIG. 2 or in another manner. In some implementations, the three internal channels 312, 314, 316 may be directly coupled with transfer tubes that extends through the housing 304 from the end opposite to the receiving end to the receiving end of the housing 304 or may be coupled with the transfer tubes in other manner to allow liquid and gas flow.

In some implementations, the housing 304 is configured to provide fluidic communication with a control system and a mass spectrometer through respective transfer tubes, e.g., the transfer tubes 206. In certain instance, the housing 304 and the probe tip 302 may be composed of biologically compatible synthetic polymers. In some implementations, the housing 304 and the probe tip 302 may be fabricated using a 3D printing process, a machining process, or another type of fabrication process.

In the example shown in FIG. 3, the sample surface 320 is a surface of a solid substrate. For example, the sample surface 320 may be a glass slide, a petri dish, or an agar plate. In some implementations, the sample surface 320 may make a liquid-tight seal with the mandrel end 306 of the probe tip 302 in order to prevent leakage of the liquid solvent from the internal reservoir 318. In some implementations, the sample surface 320 can be or include the surface of a tissue sample, or another type of biological sample. For example, the sample surface 320 can be an in-vivo or ex-vivo tissue site. In some cases, the sampling probe 300 is used during a medical procedure (e.g., during surgery) to evaluate a tissue site of a subject. In a surgery environment, the liquid solvent can be or include water, ethanol mixed with water, or another type of solvent. The sampling probe 300 may collect samples from tissue sites exposed during a surgical procedure. Cells and molecules obtained by the sampling probe 300 from the tissue site may be analyzed by a mass spectrometer to identify and classify the tissue sample, which may be used to prescribe treatment or therapy. In some cases, the sampling probe 300 is used to determine whether the tissue sample contains endometriosis tissue, to differentiate endometriosis tissue from healthy tissue, to determine surgical margin, or for another purpose.

In some aspects of operation, the liquid supply channel 312 receives the liquid solvent from an external container, guides the liquid solvent to the internal reservoir 318 at the probe tip 302, where the liquid solvent may be in direct contact with the sample surface 320, and fills at least a portion of the internal reservoir 318 with the liquid solvent. The liquid supply channel 312 may provide a first internal pathway 332 in the probe tip 302. In some implementations, the liquid solvent may be received from the external container as a part of a control system, e.g., the mechanical pumping system 212 as shown in FIG. 2.

In some implementations, the internal reservoir 318 may have a cylindrical shape and be coupled to the liquid supply channels 312. In certain examples, the liquid solvent received from the liquid supply channel 312 in the internal reservoir 318 makes direct contact with the sample surface 320. In some instances, at least a portion of the cells of the tissue sample may be suspended and molecules from the cells may be extracted into the liquid solvent. In some instances, the diameter 322 and height 324 of the internal reservoir 318 may determine the volume of the liquid solvent exposed to the sample surface 320 and performance aspects of the example system, for example a spatial resolution, limit of detection, and accuracy. In some instances, the diameter of the internal reservoir 318 of the probe tip 302 may be in a range of 1.5-5.0 mm. For example, when the diameter 322 of the internal reservoir is 2.77 mm and the height 324 of the internal reservoir 318 is 1.7 mm, the volume of a liquid solvent that is contained in the internal reservoir 318 is 10 microliter (μL). For another example, when the diameter of the internal reservoir 318 is 1.5 mm and the height 324 is 2.5 mm, the volume of a liquid solvent that is contained in the internal reservoir 318 is 4.4 μL. The internal reservoir 318 may have a different shape, aspect ratio, size or dimension.

In some instances, the liquid extraction channel 314 provides a second, distinct internal pathway 334 in the probe tip 302. In some aspects of operation, the liquid extraction channel 314 obtains a liquid sample by extracting at least a portion of the liquid solvent carrying the suspended cells or the extracted molecules from the internal reservoir 318, and guides the liquid sample to the transfer tube that is coupled to a mass spectrometer. In some implementations, the liquid sample from the internal reservoir 318 may be extracted by a vacuum pump coupled to the mass spectrometer (e.g., the mass spectrometer 220 as shown in FIG. 2). In some implementations, a low pressure created on one end of the transfer tube may facilitate liquid aspiration to drive the liquid sample from the internal reservoir 318 to the mass spectrometer through the liquid extraction channel 314.

In some implementations, the gas channel 316 provides a third, distinct internal pathway 336 in the probe tip 302. In some instances, the gas channel 316 is configured for preventing collapse of the sampling probe 300, transfer tubes and the control system during the extraction. In some instances, the gas channel 316 is open to atmosphere (e.g., air). In some instances, diameters of the liquid supply channel 312, the liquid extraction channel 314 and the gas channel 316 may be equal to 0.8 mm. Gas from the gas channel 316 may be used to push the liquid out of the liquid extraction channel 314 to the mass spectrometer.

FIG. 4 is a flow diagram showing an example process 400 for tissue analysis. In some implementations, the example process 400 may be an automated process used for analyzing tissue samples. The example process 400 may be used for qualitatively and quantitatively identification and detection of endometriosis tissue. The example process 400 may be performed, for example, by the example systems shown in FIGS. 1-3 or another type of system with additional or different components. The example process 400 may include additional or different operations, including operations performed by additional or different components, and the operations may be performed in the order shown or in another order. In some cases, operations in the example process 400 can be combined, iterated or otherwise repeated or performed in another manner.

At 402, a liquid solvent is supplied. In some implementations, the liquid solvent may be supplied to a sample surface of an in-vivo or ex-vivo tissue sample to suspend cells and/or extract molecules from the cells. In some instances, the ex-vivo tissue samples may be kept frozen and defrosted to room temperature for analysis. In some instances, after the probe tip of the sampling probe is positioned against a tissue sample, the liquid solvent is supplied to the sample surface with controlled volume and flow rate. In some implementations, a control system (e.g., the control system 210 as shown in FIG. 2) may be used to supply the liquid solvent to a sampling probe with an internal reservoir at an opening (e.g., the sampling probe 300 as shown in FIG. 3), where the liquid solvent may be in direct contact with the sample surface. In some implementations, the control system may be controlled by a computer system (e.g., the computer system 102 as shown in FIG. 1). In certain examples, the liquid solvent may be delivered to the internal reservoir of the sampling probe for a first time period. For example, a syringe pump may be used to deliver 10 μL of the liquid solvent to the internal reservoir and the syringe pump may take 2 seconds to perform the liquid solvent delivery at a flow rate of 300 μL/min.

At 404, a liquid sample is formed. In some implementations, the liquid solvent after being delivered to the internal reservoir of the sampling probe may interact with at least a portion of at the sample surface causing at least a portion of cells in the tissue samples suspended and molecules from the cells extracted into the liquid solvent. In some implementations, the liquid solvent may be allowed to interact with the tissue sample for a second time period. In some implementations, the liquid solvent may include sterile water, ethanol, methanol, acetonitrile, dimethylformamide, acetone, isopropyl alcohol, or a combination thereof. In some instances, the second time period is 3 seconds, or another duration of time may be used. In certain instances, the liquid solvent with the suspended cells and/or extracted molecules forms the liquid sample, which is contained in the internal reservoir before being extracted for mass spectrometry analysis.

At 406, the liquid sample is extracted. In some implementations, the liquid sample may include the liquid solvent with the suspended cells and/or the extracted molecules. In some implementations, extraction of the liquid sample from the sample surface may be performed by applying a pressure on one end of a transfer tube coupled to the sampling probe. In some instances, the pressure may be lower than the atmospheric pressure where the tissue sample is analyzed. In some implementations, the liquid sample may be extracted into and analyzed by a mass spectrometer. In some instances, the mass spectrometer may be an Orbitrap QE Mass Spectrometer operating under a negative ion mode with a resolving power: 120,000 and a mass accuracy <5 ppm or configured in another manner.

In certain examples, prior to being analyzed by the mass spectrometer, the liquid sample may be ionized using an electrospray ionization or another manner. In some instances, the ionized liquid sample is processed. In some implementations, the ionized liquid sample may be collected by the mass spectrometer. In some implementations, when traveling in the mass spectrometer, fragments in the ionized liquid sample may be separated and identified according to their mass-to-charge (m/z) ratio. In some examples, the ionized liquid sample may be scanned one or more times during the third time period. In some implementations, the mass spectrometer may be implemented as the mass spectrometer 220 as shown in FIG. 2 or in different manner.

In some instances, data is analyzed. In some implementations, a set of mass spectra collected by the mass spectrometer may be output to the computer system, which may be further stored and analyzed. In some implementations, a molecular profile of the liquid sample may be obtained by averaging multiple scans (e.g., the set of mass spectra) collected in the third time period by the mass spectrometer. In some instances, a mass spectral background from a blank liquid solvent may be subtracted or filtered.

In some implementations, the mass spectrometer may be a tandem mass spectrometer, where the cluster of fragment ions created by the ionization system is further filtered by selecting one or more particular fragment ions from the cluster according to m/z ratios. In some examples, the tandem mass spectrometer may filter and select the one or more particular fragment ions within a few milliseconds. In some instances, intensities of a particular fragment ion obtained during multiple scans using the tandem mass spectrometer may be then averaged.

In some implementations, the process may be automatically operated by the computer system. In some implementations, operations 402-406 may be performed in a recursive manner to obtain replicate measurement at different locations on the sample surface. In some implementations, between each replicate measurement or when switching between sampling on different sample surfaces, a cleaning process to wash the tubing and the sampling probe can be separately performed to minimize cross-contamination.

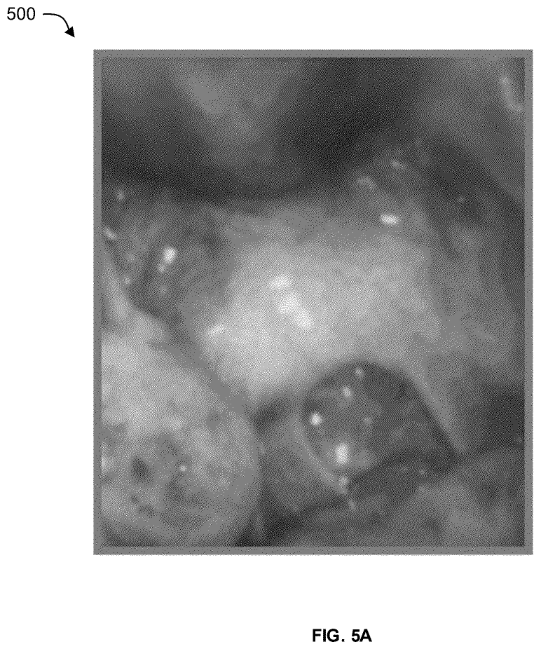

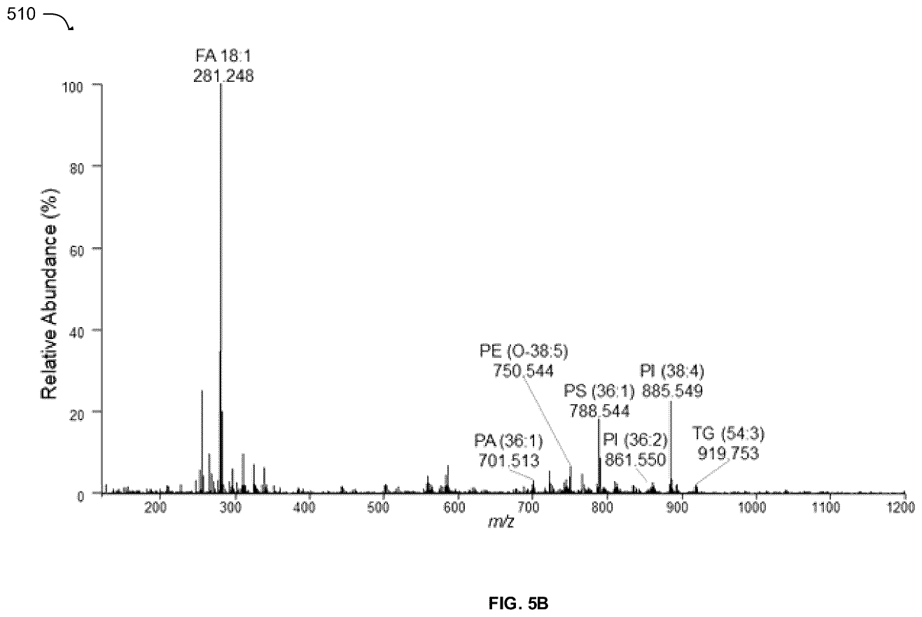

FIG. 5A is an optical image 500 of an in-vivo endometriotic lesion. FIG. 5B is an example mass spectrum 510 of an ex-vivo endometriotic tissue sample taken from the cul-de-sac of a patient. The example mass spectrum 510 can be obtained, e.g., by the example systems shown in FIGS. 1-3. In some examples, the system may include a sampling probe which may include a probe tip with an internal reservoir (e.g., the sampling probes 202, 300 as shown in FIGS. 2-3). In some implementations, the system includes a mass spectrometer for sample analysis. In some implementations, the mass spectrometer is a ThermoFisher Q Exactive Orbitrap mass spectrometer operating in negative ion modes in a mass-to-charge (m/z) range of 120-1000 with a resolving power of 120,000. In some implementations, high mass accuracy measurements are used to identify molecular ions.

In some instances, after the probe tip of the sampling probe is positioned against the ex-vivo tissue sample, for example on a glass slide, a fixed volume of a liquid solvent, e.g., water, for suspending cells from the tissue sample and/or extracting molecules from the cells of the tissue sample is delivered to the internal reservoir of the sampling probe. In some instances, the fixed volume of the liquid solvent in the internal reservoir is kept in direct contact with the tissue sample for a time period, e.g., extraction time of 3-10 seconds. After the time period, a liquid sample is obtained and transferred from the internal reservoir to the mass spectrometer for analysis. In some instances, replicates may be collected on the same tissue sample at different locations or on different tissue samples with the same type.

In some implementations, a variety of molecular features in the example mass spectrum 510 corresponding to the molecules extracted from the tissue sample may be used to detect endometriosis. For example, the molecules may include metabolites, free fatty acids (FA), glycerphosphoserines (PS), glycophosphoinositols (PI), glycerophosphoethanolamines (PE), phosphatidic acid (PA) and another molecule.

Mass spectrometry data of the endometriotic tissue sample provided unique peaks compared with healthy tissue. In the example shown in FIG. 5B, a higher relative abundance of FA 18:1 (m/z=281.248), PA (36:1) (m/z=701.513), PE (O-38:5) (m/z=750.544), PS (36:1) (m/z=788.544), PI (36:2) (m/z=861.550), and PI (38:4) (m/z=885.549) is observed. In some implementations, these peaks are used as the molecular features for training a statistical classification model, which are weighted toward classification of endometriosis tissue samples.

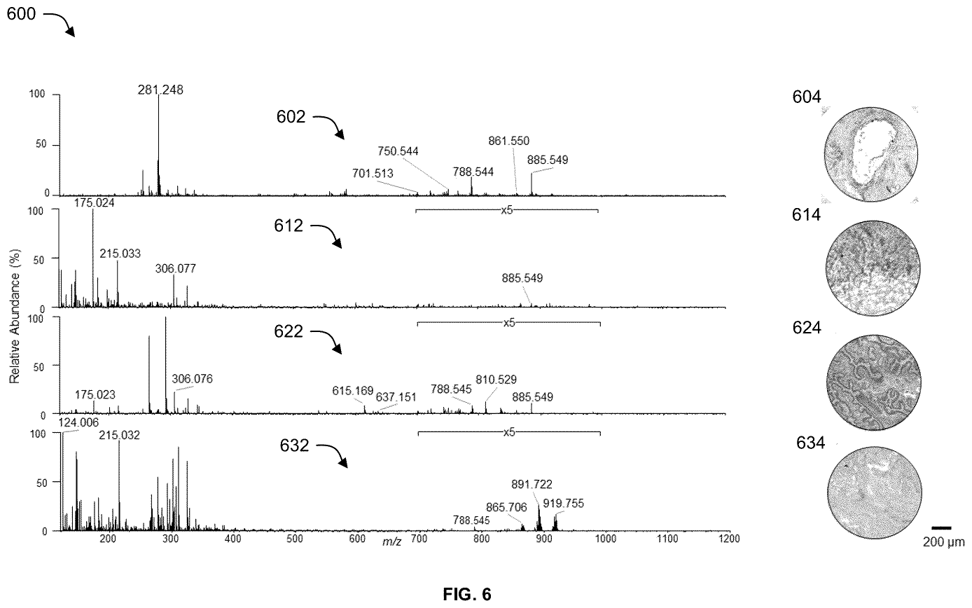

FIG. 6 are example mass spectra and respective post-analysis histopathological images of various tissue samples. The example mass spectra shown in FIG. 6 are obtained from an endometriosis tissue sample taken from the cul-de-sac, an ovary tissue sample, a fallopian tube mucosa sample, and a soft tissue sample. The example mass spectra 600 are obtained, e.g., by a system such as the example systems shown in FIGS. 1-3. In some examples, the system may include a sampling probe which may include a probe tip with an internal reservoir (e.g., the sampling probes 202, 300 as shown in FIGS. 2-3). The example mass spectra 600 are obtained following a tissue analysis process as described in FIG. 4. In some implementations, the probe tip and transfer tubes may be cleaned thoroughly by flushing solvent or may be replaced when switching between different tissue samples to prevent contamination.

As shown in FIG. 6, qualitative differences in molecular profiles of the tissue samples are observed in the mass spectra. For example, six significant peaks are observed at m/z=281.248, m/z=701.513, m/z=750.544, m/z=788.544, m/z=861.550, m/z=885.549 in the first example mass spectrum 602 obtained on the endometriosis tissue sample; three significant peaks at m/z=175.023, m/z=215.033, and m/z=306.077 are observed in a second example mass spectrum 612 obtained on the ovary tissue sample. In a third example mass spectrum 622 obtained on the fallopian tube mucosa sample, two significant peaks at m/z=175.023, and m/z=306.077 are observed. In a fourth example mass spectrum 632 obtained on the soft tissue sample, five significant peaks at m/z=124.006, m/z=215.032, m/z=865.706, m/z=891.722 and m/z=919.755 are observed. In some implementations, these peaks are used as molecular features for training a statistical classification model.

In some implementations, to build a statistical model (e.g., classifier), the tissue samples are further sectioned and validated using pathological evaluation. Regions on respective tissue samples where the mass spectra are collected are then prepared for Hematoxylin and eosin stain (e.g., H&E stain) and pathological evaluation. Microscopic images of the regions on the respective tissue samples with H&E stains are shown in 604, 614, 624, and 634.

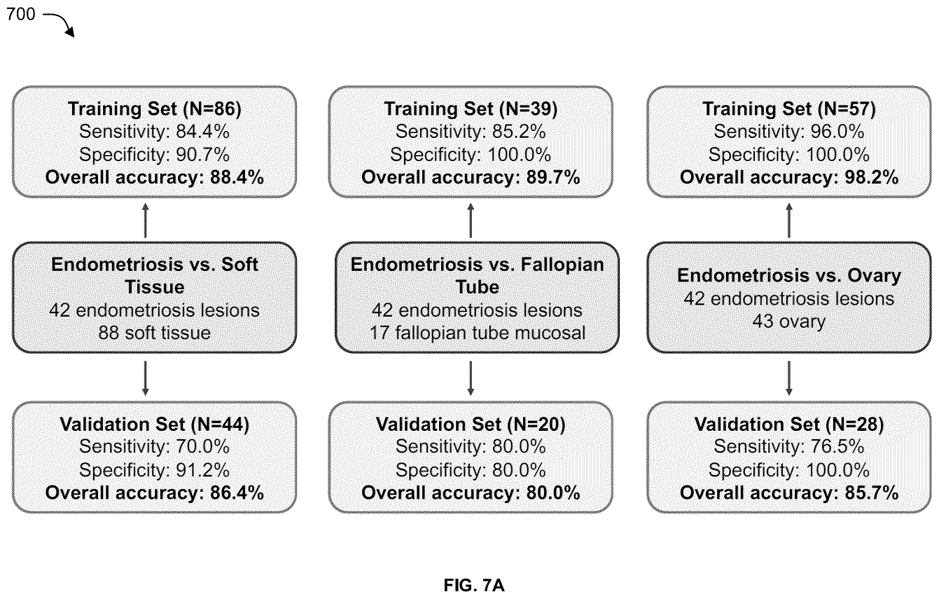

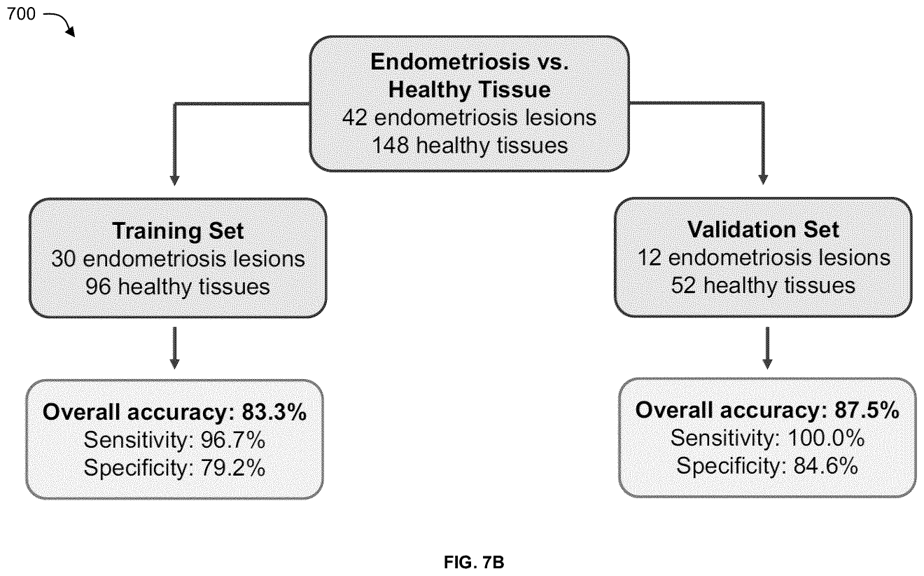

FIGS. 7A-7B are diagrams 700 showing performance of a statistical classification model. Mass spectra of 190 tissue samples including 42 endometriosis tissue samples, and 148 blank tissue samples from healthy abdominal tissues are used. Particularly, the 148 blank tissue samples include 17 fallopian tube mucosal samples, 43 ovary tissue samples, and 88 soft tissue samples. The mass spectra of the 190 tissue samples are obtained by a system that includes a mass spectrometer. In some examples, the system includes a sampling probe which may include a probe tip with an internal reservoir (e.g., the sampling probes 202, 300 as shown in FIGS. 2-3).

As shown in FIG. 7A, the 190 tissue samples are divided into three training sets and three validation sets. In a first molecular classification process, a first training set includes 32 endometriosis tissue samples and 54 soft tissue samples; and a first validation set includes 10 endometriosis tissue samples and 34 soft tissue samples. In a second molecular classification process, a second training set includes 27 endometriosis tissue samples and 12 fallopian tube mucosal samples; and a second validation set includes 15 endometriosis tissue samples and 5 fallopian tube mucosal samples. In a third molecular classification process, a third training set includes 25 endometriosis tissue samples and 32 ovary tissue samples; and a third validation set includes 17 endometriosis tissue samples and 11 ovary tissue samples.

In some implementations, an accuracy, a sensitivity, and a specificity are defined as shown below.

$$Accuracy = \frac{True Positive + True Negative}{True Positive + False Positive + True Negative + False Negative}$$

$$Sensitivity = \frac{True Positive}{True Positive + False Negative}$$

$$Specificity = \frac{True Negative}{False Positive + True Negative}$$

As shown in FIG. 7A, on the first training set in the first molecular classification process to differentiate the endometriosis tissue samples from the soft tissue samples, the statistical classification model produces an overall accuracy of 88.4%, a sensitivity of 84.4% and a specificity of 90.7%; and on the first validation set, the statistical classification model produces an overall accuracy of 86.4%, a sensitivity of 70.0% and a specificity of 91.2%. On the second training set in the second molecular classification process to differentiate the endometriosis tissue samples from the fallopian tube mucosal samples, the statistical classification model produces an overall accuracy of 89.7%, a sensitivity of 85.2% and a specificity of 100.0%; and on the second validation set, the statistical classification model produces an overall accuracy of 80.0%, a sensitivity of 80.0% and a specificity of 80.0%. On the third training set in the third molecular classification process to differentiate the endometriosis tissue samples from ovary tissue samples, the statistical classification model produces an overall accuracy of 98.2%, a sensitivity of 96.0% and a specificity of 100.0%; and on the third validation set, the statistical classification model produces an overall accuracy of 85.7%, a sensitivity of 76.5% and a specificity of 100.0%.

As shown in FIG. 7B, in a fourth molecular classification process to differentiate the endometriosis tissue samples from the healthy tissue samples, the 190 tissue samples are divided into a fourth training set and a fourth validation set. The fourth training set includes 30 endometriosis tissue samples and 96 healthy tissue samples including 29 ovary tissues, 58 soft tissues and 9 fallopian tube mucosal samples. The fourth validation set includes 12 endometriosis tissue samples and 52 healthy tissue samples including 14 ovary tissues, 30 soft tissues and 8 fallopian tube mucosal samples.

As shown in FIG. 7B, on the fourth training set in the fourth molecular classification process, the statistical classification model produces an overall accuracy of 84.1%, a sensitivity of 96.7% and a specificity of 80.2%. On the fourth validation set in the fourth molecular classification process, the statistical classification mode produces an overall accuracy of 87.5%, a sensitivity of 100.0% and a specificity of 84.6%.

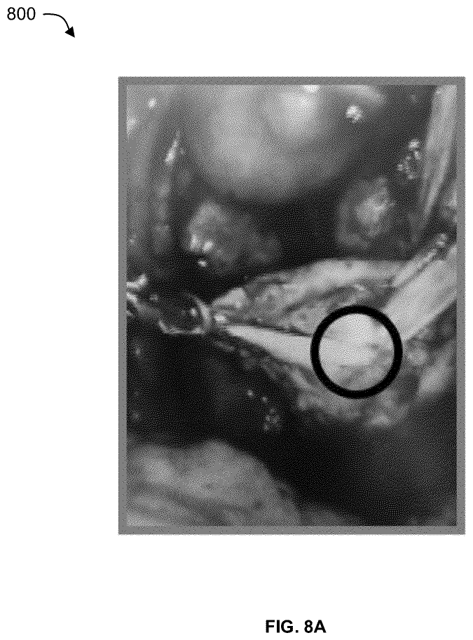

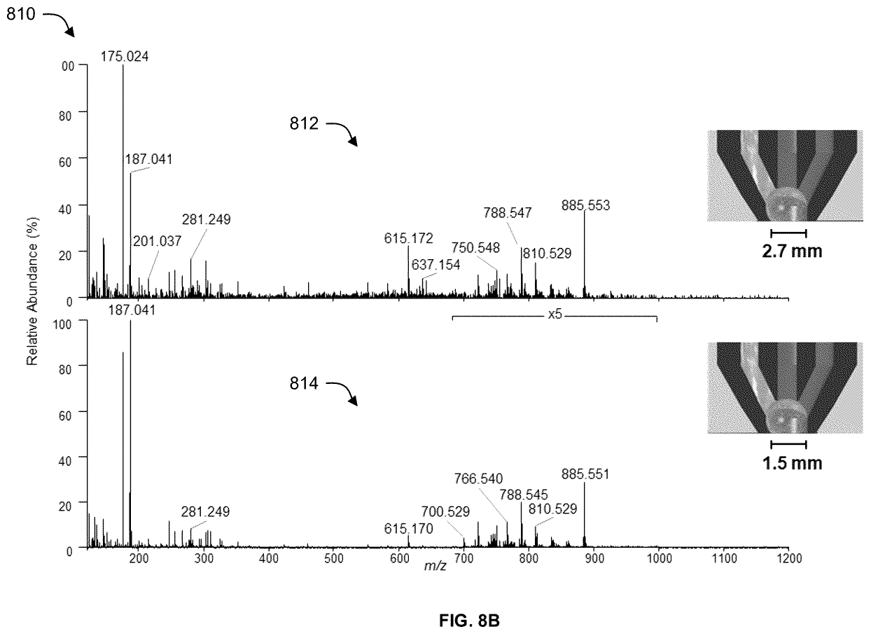

FIG. 8A is an optical image 800 of an in-vivo endometriosis lesion on a right ovary of a patient. FIG. 8B are example mass spectra 810 collected on an ex-vivo endometriosis tissue sample taken from a right ovary. In some implementations, molecular profiles of the endometriosis tissue sample are used for determining the size effect of a probe tip to the mass spectrum. In some instances, the molecular profiles of the endometriosis tissue sample may be obtained using probe tips of different sizes. A first probe tip with a reservoir diameter of 2.7 mm and a second probe tip with a reservoir diameter of 1.5 mm are compared. Molecular features in the mass spectra 812, 814 shown in FIG. 8B obtained using the two different probe tips are consistent and no significant differences (e.g., relative abundance of peaks at respective m/z values) are observed. In some implementations, a higher spatial resolution may be achieved by using a probe tip with a smaller reservoir diameter. In some instances, the higher spatial resolution allows for specimen mapping and determining surgical margin.

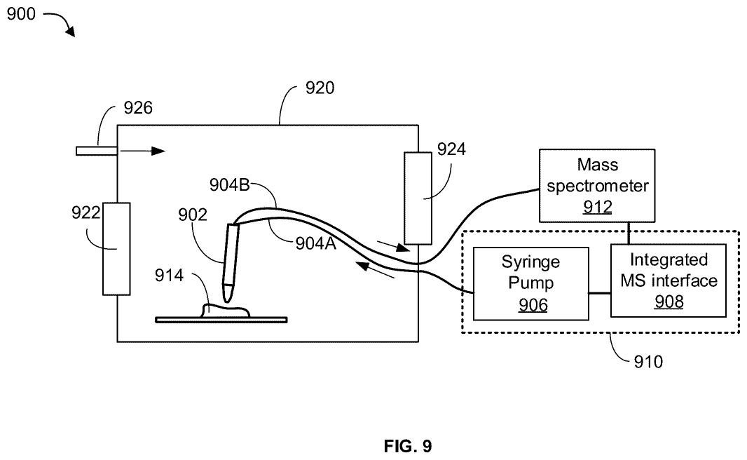

FIG. 9 is a block diagram showing aspects of an example system 900. In some implementations, the example system 900 is used for tissue analysis in a CO₂ atmosphere. The example system 900 includes a sampling probe 902, transfer tubes 904A, 904B, a control system 910, and a mass spectrometer 912. The control system 910 includes a syringe pump 906 and an integrated MS interface 908. In some instances, the example system 900 may include aspects that are similar to the example systems as shown in FIGS. 1-3. The example system 900 further includes a glovebox 920. In some instances, the sampling probe 902 resides and can be operated in the glovebox 920 under controlled gas composition, temperature, and pressure. In some instances, the example system 900 may include additional or different components, and the components may be arranged as shown or in another manner.

As shown in FIG. 9, the glovebox 920 includes built-in gloves 922 and a transfer door 924. In some instances, the built-in gloves 922 are positioned on sidewalls of the glovebox 920 allowing a user to perform analysis tasks on a tissue sample 914 without breaking containment. In some instances, at least a portion of the sidewalls of the glovebox 920 is transparent allowing the user to see through when performing the analysis tasks. The transfer door 924 is positioned on one of the sidewalls of the glovebox 920 to allow loading and unloading the tissue sample 914 into or out of the glovebox 920. In some instances, the glovebox 920 can be purged via one or more gas inlets 926. In some instances, one of the one or more gas inlets 926 can receive carbon dioxide gas or another type of gas from a gas tank at a higher pressure. In some examples, the glovebox 920 may be also pumped to remove substance, such as particles, water, or oxygen, to create a controlled atmosphere. In some instances, the glovebox 920 may be a flexible glovebox, a plastic glovebox, a metal glovebox, or another type of glovebox. In some instances, the example system 900 can be operated under positive pressure provided by the glovebox 920.

As shown in FIG. 9, the sampling probe 902 of the example system 900 is positioned inside of the glovebox 920 and fluidically coupled with the syringe pump 906 and the mass spectrometer 912 via respective transfer tubes 904A, 904B. In some instances, the respective transfer tubes 904A, 904B run through one of the sidewalls of the glovebox 920 without breaking the containment to provide a liquid solvent to the sampling probe 902 from the syringe pump 906 and to extract a liquid sample to the mass spectrometer 912.

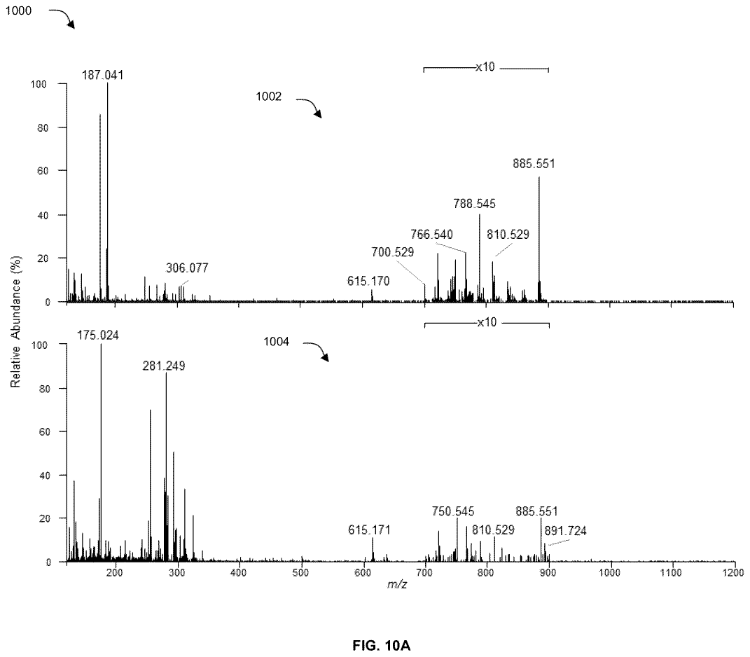

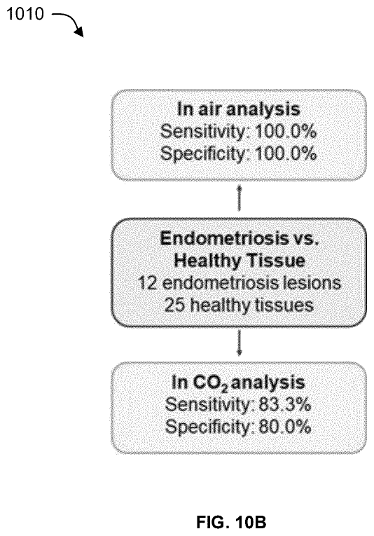

FIG. 10 shows example mass spectra 1000 collected on an ex-vivo endometriosis tissue sample taken from a right ovary. In some instances, the mass spectra 1000 are collected by operation of a mass spectrometer processing a sample obtained from tissue sample in air and in a glovebox. The glovebox is implemented as the glovebox 920 shown in FIG. 9 filled with carbon dioxide gas. In some instances, the system may be implemented as shown in FIGS. 1-3 and 9. In some examples, the system may include a sampling probe which may include a probe tip with an internal reservoir (e.g., the sampling probes 202, 300 as shown in FIGS. 2-3). In some instances, the example mass spectra 1000 are obtained according to a tissue analysis process, e.g., the tissue analysis process 400 shown in FIG. 4 or in another manner.

As shown in FIG. 10, qualitative differences in molecular profiles in the mass spectra of the endometriosis tissue sample collected in different environment can be observed. For example, in a first mass spectrum 1002, a higher relative abundance of PS 36:1 (m/z=788.545) and PI 38:4 (m/z=885.551) can be observed on the endometriosis tissue sample when analyzed in air. A higher relative abundance FA 18:1 (m/z=281.249) and PE-O 38:5 (m/z=750.545) can be observed in a second mass spectrum 1004 obtained on the same tissue sample when analyzed in the glovebox in a CO₂ atmosphere.

FIG. 10B is a diagram 1010 showing performance of a statistical classification model. As shown in FIG. 10B, 37 tissue samples including 12 endometriosis tissue samples and 25 healthy tissue samples are measured in air and in a CO₂ atmosphere. Mass spectra of the 37 tissue samples are used to evaluate the statistical classification model. The statistical classification model produces a sensitivity of 100% and a specificity of 100.0% on the tissue samples analyzed in air. The statistical classification model produces a sensitivity of 83.3% and a specificity of 80.0% on the tissue samples analyzed in the CO₂ atmosphere.

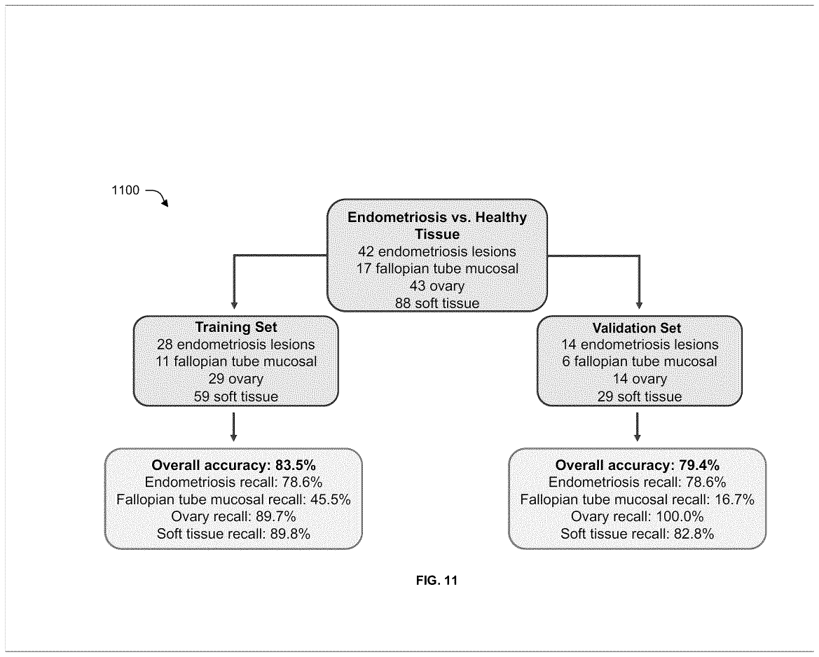

FIG. 11 is a diagram 1100 showing performance of a statistical classification model. Mass spectra of 190 tissue samples including 42 endometriosis tissue samples, and 148 healthy tissue samples are used to train the statistical classification model and evaluate the performance. The 148 healthy tissue samples include 17 fallopian tube mucosal samples, 43 ovary tissue samples, and 88 soft tissue samples. The 190 samples are divided into a training set and a validation set. The training set includes 28 endometriosis lesion samples, 11 fallopian tube mucosal samples, 29 ovary tissue sample, and 59 soft tissue sample. The validation set includes 14 endometriosis lesion samples, 6 fallopian tube mucosal samples, 14 ovary, and 29 soft tissue samples. The example mass spectra are generated by a mass spectrometer processing samples obtained as described with respect to FIGS. 1-3. In some examples, the system includes a sampling probe which may include a probe tip with an internal reservoir (e.g., the sampling probes 202, 300 as shown in FIGS. 2-3).

In some implementations, a recall is defined as shown below.

$$Recall = \frac{True Positive}{True Positive + False Negative}$$

On the tissue samples in the training set, the statistical classification model produces an endometriosis recall of 78.6%, a fallopian tube recall of 45.5%, an ovary recall of 89.7% and a soft tissue recall of 89.8%, resulting an overall accuracy of 83.5%. On the tissue samples in the validation set, the statistical classification model produces an endometriosis recall of 78.6%, a fallopian tube recall of 16.7%, an ovary recall of 100.0% and a soft tissue recall of 82.8%, resulting an overall accuracy of 79.4%.

Some of the subject matter and operations described in this specification can be implemented in digital electronic circuitry, or in computer software, firmware, or hardware, including the structures disclosed in this specification and their structural equivalents, or in combinations of one or more of them. Some of the subject matter described in this specification can be implemented as one or more computer programs, i.e., one or more modules of computer program instructions, encoded on a computer storage medium for execution by, or to control the operation of, data-processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media.

Some of the operations described in this specification can be implemented as operations performed by a data processing apparatus on data stored on one or more computer-readable storage devices or received from other sources.

The term “data-processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program, or in multiple coordinated files (e.g., files that store one or more modules, sub programs, or portions of code). A computer program can be deployed to be executed on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

In a general aspect of what is described above, endometriosis tissue is identified using mass spectrometry.

In a first example, a fixed or discrete volume of a solvent is applied to a tissue site including possible endometriosis tissue in the subject. The applied solvent is collected to obtain a liquid sample. The liquid sample is subjected to mass spectrometry analysis.

Implementations of the first example may include one or more of the following features. The tissue site is identified as endometriosis tissue versus healthy tissue. The sample is collected in a substantially CO₂ atmosphere. The endometriosis tissue is endometriosis on the cul-de-sac or ovarian endometriosis. The tissue is assessed by histological analysis. The tissue site is on the fallopian tube or the ovary. The subject has previously been assessed by a pelvic exam, ultrasound or MRI. The tissue site is identified as endometriosis tissue versus soft tissue; endometriosis tissue versus fallopian tube tissue or endometriosis tissues versus ovary tissue.

Implementations of the first example may include one or more of the following features. A mass-to-charge (m/z) ratio of about 132.0; 281.2; 307.2; 700.5; 701.5; 750.5; 788.5; 861.5; 885.5; and/or 892.7 is measured. A mass-to-charge (m/z) ratio of about 152.5; 175.0; 187.0; 201.0; 210.10; 261.0; 281.2; 615.2; 616.2; 637.2; 700.5; 701.5; 729.5; 750.5; 766.5; 788.5; 810.5; 861.5; 885.6; and/or 892.7 is measured. A mass-to-charge (m/z) ratio of about 152.5; 281.2; 312.2; 480.3; 701.5; 718.5; 729.6; 750.5; 766.5; 788.5; 859.5; 861.5; 861.6; 885.5; and/or 919.8 is measured. A mass-to-charge (m/z) ratio of about 175.0; 210.1; 281.2; 373.0; 615.2; 700.5; 747.5; 750.5; 771.5; 773.5; 810.5; 836.5; 884.5; 885.5; and/or 891.7 is measured. A mass-to-charge (m/z) ratio of about 132.0; 175.0; 187.0; 195.0; 281.2; 306.1; 615.2; 700.5; 766.5; 788.5; 810.5; and/or 885.5 is measured. A mass-to-charge (m/z) ratio of about 281.2; 330.2; 480.3; 672.5; 701.5; 750.5; 788.5; 859.5; 861.5; 885.5; and/or 892.7. A mass-to-charge (m/z) ratio of about 343.03; 401.99; 403.9; 447.0; and/or 771.5 is measured. The amount of lactate, gluconate, arachidonic acid, ascorbate, oleic acid, aspartate, glutathione, glycerophosphoethanolamine, glycerophosphoinisitol, triacylglycerol, or glycerophosphoserine in the sample is measured.

Implementations of the first example may include one or more of the following features. The fixed or discrete volume of a solvent is not applied as a spray. The fixed or discrete volume of a solvent is applied as a droplet. The fixed or discrete volume of a solvent is applied through the cannula of a surgical instrument. The surgical instrument is a laparoscope. The surgical instrument is a trocar needle. The trocar is an 8 mm trocar. The surgical instrument is a biopsy guide. The surgical instrument is manually operated. The surgical instrument is robotic.

Implementations of the first example may include one or more of the following features. A dye is applied to the tissue site. The tissue site is imaged. The tissue site is imaged by visual, fluorescent, US, CT, MRI or OCT imaging. The cannulas included in a probe has a distal probe end and the distal probe end includes a shutter that can be closed to prevent fluid from passing out of the cannula of the probe. The shutter is a balloon that can be inflated to prevent fluid communication outside of the probe. The balloon can be inflated with a gas. The shutter is a door than can be closed to prevent fluid communication outside of the probe.

Implementations of the first example may include one or more of the following features. The fixed or discrete volume of a solvent is applied at using a mechanical pump to move the solvent through a solvent conduit. The applied solvent is collected by applying a negative pressure to pull the sample into a collection conduit and/or applying a gas pressure to push the sample into a collection conduit. The applied solvent is collected by applying a negative pressure to pull the sample into a collection conduit and applying a positive pressure to push the sample into a collection conduit. The solvent is applied through a solvent conduit that is separate from the collection conduit. The gas pressure is applied through a gas conduit that is separate from the solvent conduit and the collection conduit. A gas pressure is applied to push the sample into a collection conduit includes applying a pressure of less than 100 psig. The method produces no detectable physical damage to the tissue. The method does not involve application of ultrasonic or vibrational energy to the tissue.

Implementations of the first example may include one or more of the following features. The solvent is sterile. The solvent is pharmaceutically acceptable formulation. The solvent is an aqueous solution. The solvent is sterile water. The solvent consists essentially of water. The solvent includes from about 1 to 20% of an alcohol. The alcohol includes ethanol. The discrete volume of solvent is between about 0.1 and 100 μL. The discrete volume of solvent is between about 1 and 50 μL.

Implementations of the first example may include one or more of the following features. The applied solvent is collected between 0.1 and 30 seconds after the liquid solvent is applied. The applied solvent is collected between 1 and 10 seconds the liquid solvent is applied. The tissue site is an internal tissue site that is being surgically assessed. Tissue identified as endometriosis tissue is resected. A plurality liquid samples is collected from a plurality of tissue sites. The liquid samples are collected with a probe. The probe is washed between collection of the different samples. The probe is disposable and is changed between collection of the different samples. The probe includes a collection tip and further comprising ejecting the collection tip from the probe after the liquid samples are collected. The plurality of tissue sites includes 2, 3, 4, 5, 6, 7, 8, 9 or 10 tissues sites.

Implementations of the first example may include one or more of the following features. The mass spectrometry includes ambient ionization MS. A profile corresponding to the tissue site is determined. The profile is compared to a reference profile to identify tissue sites that include endometriosis tissue. Tissue sites that are identified to include endometriosis tissue are resected. The tissue sites are resected by laser ablation. The tissue type at different sites are determined.

In a second example, a solvent is supplied to a tissue site via a first channel of a sampling probe. The solvent is supplied to the tissue site in vivo during a medical procedure. The solvent interacts with the tissue site to form a sample in the sampling probe. The sample is transferred from the sampling probe via a second channel of the sampling probe. The sample is transferred to a mass spectrometer. By operation of the mass spectrometer, the sample is processed to produce mass spectrometry data. The mass spectrometry data is analyzed to identify whether the tissue site includes endometriosis tissue.

Implementations of the second example may include one or more of the following features. The tissue site is categorized as one of endometriosis tissue, soft tissue, fallopian tube mucosa, or ovary tissue. The tissue site is identified as endometriosis tissue or healthy tissue. The solvent is supplied to the tissue site in vivo during a laparoscopic procedure, and the sampling probe includes at least one of a laparoscope, a trocar needle, or a biopsy guide. Carbon dioxide is introduced to an atmosphere of the tissue site, while the solvent is supplied to the tissue site in a substantially carbon dioxide atmosphere.

In a third example, a system includes a sampling probe, a mass spectrometer and a computer system. The sampling probe includes a first channel and a second channel. The sampling probe is configured to supply a solvent to a tissue site in vivo during a medical procedure. The solvent is supplied through the first channel and interacts with the tissue site to form a sample in the sampling probe. The sample is transferred from the sampling probe via the second channel. The mass spectrometer is configured to receive the sample and process the sample to produce mass spectrometry data. The computer system is configured to analyze the mass spectrometry data to identify whether the tissue site includes endometriosis tissue.

Implementations of the third example may include one or more of the following features. The tissue site is categorized as one of endometriosis tissue, soft tissue, fallopian tube mucosa, or ovary tissue. The tissue site is identified as endometriosis tissue or healthy tissue. The solvent is supplied to the tissue site in vivo during a laparoscopic procedure, and the sampling probe includes at least one of a laparoscope, a trocar needle, or a biopsy guide. Carbon dioxide is introduced to an atmosphere of the tissue site, while the solvent is supplied to the tissue site in a substantially carbon dioxide atmosphere. The sampling probe further includes a third channel and a reservoir. The solvent interacts with the tissue site to form a sample in the reservoir of the sampling probe, and the first, second and third channels are in fluid communication with the reservoir.

In a fourth example, mass spectrometry data generated by a mass spectrometer processing a sample collected from a tissue site in vivo during a medical procedure is received. The mass spectrometry data is analyzed to identify whether the tissue site includes endometriosis tissue.

In a fifth example, a computer-readable medium stores instructions that are operable when executed by a data-processing apparatus to perform one or more operations of the fourth example.

Implementations of the fourth or the fifth example may include one or more of the following features. The tissue site is categorized as one of endometriosis tissue, soft tissue, fallopian tube mucosa, or ovary tissue. The tissue site is identified as endometriosis tissue or healthy tissue. The solvent is supplied to the tissue site in vivo during a laparoscopic procedure, and the sampling probe includes at least one of a laparoscope, a trocar needle, or a biopsy guide. The solvent is supplied to the tissue site in a substantially carbon dioxide atmosphere.

While the compositions and methods of this disclosure have been described in terms of example embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the present disclosure as defined by the appended claims.

What is claimed is:

1. A sampling probe comprising:
a first channel, a second channel, a third channel, and an internal reservoir;
wherein the first channel, the second channel, and the third channel are each in fluid communication with the internal reservoir;
wherein the first channel, the second channel, and the third channel each comprise a respective terminus at the internal reservoir;
wherein the first channel is configured to provide fluid communication between a solvent chamber and the internal reservoir;
wherein the second channel is configured to provide fluid communication between a mass spectrometer and the internal reservoir;
wherein the third channel has an open port configured to receive air from the atmosphere surrounding the sampling probe, wherein the third channel is configured to provide fluid communication between a gas supply and the reservoir, the gas supply being the atmosphere surrounding the sampling probe;
wherein the sampling probe is configured to:
supply a fixed or discrete volume of solvent to a tissue site via the first channel to the internal reservoir,
hold the fixed or discrete volume of solvent in the internal reservoir in direct contact with the tissue site for a period of time such that the fixed or discrete volume of solvent interacts with the tissue site to form a liquid sample in the internal reservoir of the sampling probe;
supply air to the internal reservoir of the sampling probe via the third channel; and
transfer the liquid sample from the sampling probe via the second channel by extracting the liquid sample form the internal reservoir via the second channel and transferring the liquid sample to a mass spectrometer via the second channel.

2. The sampling probe of claim 1, wherein the fixed or discrete volume of solvent is not applied as a spray.

3. The sampling probe of claim 1, wherein the fixed or discrete volume of solvent is a droplet.

4. The sampling probe of claim 1, wherein the sampling probe is within the cannula of a surgical instrument.

5. The sampling probe of claim 1, wherein the surgical instrument is a laparoscope, a trocar needle, or a biopsy guide.

6. The sampling probe of claim 5, wherein the surgical instrument is manually operated or robotic.

7. The sampling probe of claim 1, wherein the sampling probe further comprises a shutter that is configured to be closed to prevent fluid from passing out of the internal reservoir of the sampling probe.

8. The sampling probe of claim 7, wherein the shutter is a balloon that is configured to be inflated to prevent fluid communication outside of the sampling probe.

9. The sampling probe of claim 7, wherein the shutter is a door that is configured to be closed to prevent fluid communication outside of the sampling probe.

10. A method for assessing tissue samples from a subject, the method comprising:
(a) applying a fixed or discrete volume of a solvent via the first channel of the sampling probe of claim 1 to a tissue site including possible endometriosis tissue in the subject;
(b) collecting the applied solvent via the third channel of the sampling probe to obtain a liquid sample;
(c) transferring the liquid sample to a mass spectrometer via the second channel of the sampling probe; and
(d) subjecting the liquid sample to mass spectrometry analysis to produce mass spectrometry data.

11. The method of claim 10, further comprising analyzing the mass spectrometry data to determine whether the tissue site comprises endometriosis tissue.

12. The method of claim 10, wherein the endometriosis tissue is endometriosis on the cul-de-sac or ovarian endometriosis.

13. The method of claim 10, wherein the tissue site is identified as endometriosis tissue versus soft tissue; endometriosis tissue versus fallopian tube tissue; or endometriosis tissues versus ovary tissue.

14. The method of claim 10, wherein the method produces no detectable physical damage to the tissue.

15. The method of claim 10, wherein the mass spectrometry comprises ambient ionization MS.

16. The method of claim 10, wherein subjecting the sample to mass spectrometry analysis comprises determining a profile corresponding to the tissue site.

17. The method of claim 10, further comprising comparing the profile to a reference profile to identify tissue sites that include endometriosis tissue.

18. The method of claim 10, further comprising resecting tissue sites that are identified to include endometriosis tissue.

19. The method of claim 10, wherein analyzing the mass spectrometry data comprises categorizing the tissue site as one of endometriosis tissue, soft tissue, fallopian tube mucosa, or ovary tissue.

20. The method of claim 10, wherein analyzing the mass spectrometry data comprises identifying the tissue site as endometriosis tissue or healthy tissue.
